(19) United States
(12) Patent Application Publication
(10) Pub. No.: US 2020/0054688 A1
(43) Pub. Date: Feb. 20, 2020

(54) Method for Precise Identification, Targeting and Delivery of Directed Therapies to Domesticated Animals Using Bacteria to Bind and Destroy Cancerous Cells

(71) Applicant: Richard Postrel, Miami Beach, FL (US)

(72) Inventor: Richard Postrel, Miami Beach, FL (US)

(21) Appl. No.: 16/104,168

(22) Filed: Aug. 17, 2018

(51) Int. Cl. A61K 35/74; A61K 38/17

(52) U.S. Cl. CPC A61K 35/74; A61K 38/1709

(57) ABSTRACT

This invention teaches systems and methods for identifying, targeting and destroying cancer cells domesticated organism such as a pet or zoo animal. As cells progress from a normal to a cancerous state their accelerated metabolic rates and adapted pathways generate a higher heat signature that serves as a targeting beacon for a specialized cell killing vector. Suitable vectors include modified or adapted viruses, modified or adapted intracellular bacteria and/or engineered liposomes. Especially preferred is the bacterial vector because of its ease of production. The bacterial vector is selectively targeted to recognize cells whose temperature is slightly elevated and ambient pH suppressed due to cancer related alterations to metabolism. An additional targeting feature, such as recognition of the MCT4 transmembrane protein exaggeratively expressed on the cancer cell outer membrane, may provide additional targeting specificity. Embodiments featuring facultative extracellular and intracellular growth capable bacteria have the preferred feature that culture conditions for producing the vector can be optimized solely for the one organism and need not be compromised to support or optimize host cell maintenance.

### BACKGROUND

Cancer is not a single disease, but rather a class of diseases which are in a perpetual state of change and development. Each complex macroorganism is challenged by cancer millions of times in its lifetime. The difference between becoming a cancer victim and remaining healthy is that in most cases the mammalian immune system and associated defense mechanisms is sufficient to restore or rebalance its biochemistry to prevent undesirable and opportunistic contagions from dominating cellular growth and behavior.

The present invention exploits characteristics inherent in developing cancers to target and destroy cancer cells. Anti-cancer cell structures may be synthesized from biomolecules and/or may be synthesized using microbiological tools such as viruses, bacteria, etc. The present invention spotlights prokaryotic, e.g., bacterial organisms adapted to destroy growing cancer cells. A primarily targeted organism is any organism of value, for example, domesticated or zoo animals, including, but not limited to: dogs, cats, birds, other pets, rare or endangered species, zoo animals, etc.

Each cancerous cell presents an early onset bio-nanomarker in the form of one or more metabolic differentials that have shifted to support the massively enlarged number of chemical reactions/interactions necessary to support the enhanced replication, or simply “hyperproliferation” that is characteristic of cells of the cancer group. Although different cancers may appear in disparate tissues and cancer cells may migrate from one tissue to another, at their root each cancer cell cohort involves a shift in normal metabolism from a lower to a higher metabolic rate, this shift being characteristic of hyperproliferating cancerous cells. As a cell transitions to become cancerous, it alters its metabolic pathways in various ways; down-regulating several, up-regulating others, possibly reinvigorating pathways used at an earlier time, for example during fetal development and turning off still others entirely.

As an example of a changed metabolic requirement, each time a cell divides it requires its own set of nucleic acids to construct a second complete genome. To accomplish this, the nucleic acid production pathway must be up-regulated. But the up-regulation of one pathway requires diverting nutrient availability within the cell to deprive other pathways of their normal resource pools favoring transformation towards a more opportunistic cancerous supportive metabolic function. Outcomes of these metabolic shifts include an increased release of H⁺ with a resultant drop in pH and increased release of small carbon containing molecules. Since all cancer cells are on their face abnormal, their activities, i.e., metabolism, will present diverse metabolic pictures, with the commonality of pathways supporting hyperproliferation. In view of these considerations, cancer can be thought of as a single disease—inappropriate hyperproliferation—but with several modes of expression that are supra-dependent on the initial metabolic status of the cell and stresses or pressures that make or cause the metabolic changes to occur that are necessary to support hyperproliferation. These metabolic changes that develop in all cancer cells as they transform can serve as markers for developed or developing cancers and when properly recognized and exploited as targets to aid in the cells' destruction. Therefore, these elevated temperatures and decreased pH integral to the cancer process can be targeted therapeutically.

Cancer derives as an offshoot of mismatches in copying DNA (mutations) or opportunistic set of circumstances that supersedes cells' normal inhibition of growth of like neighboring cells. Evolution, survival of the fittest, requires differences between individuals of the species so that better suited members of the species survive to produce a next generation. Mismatched DNA are the means through which individual differences are possible. So it can be said that cancer is actually a result of evolution and that occasional mutations are advantageous to survival of the species. However, those that are cancerous, as with most mutations, are not.

We have seen that mutation events tend to increase when stress is present. This makes evolutionary sense that in times where (genetic) experimentation is desired to handle a changing (stressing) situation, tools to cope with and overcome the stress would be of more use. So every time a cell makes a copy of itself, evolution dictates that, depending on the level of difficulty the living thing is undergoing, minor changes in the genetic material (mutations) will occur in response.

One area where mutation markers are well documented is in the study of Inborn Errors of Metabolism (IEMs). Over a century ago Archibald Garrod popularized the concept that human diseases were inheritable in accordance with rules of Mendelian genetics. More than 500 IEMs have now been catalogued including many that are apparently often symptom free to a casual observer, but perhaps may have been beneficial in the past using alternative metabolic paths or specific substrate sources. Other current IEMs may lead to early death and thus would be removed from the gene pool as vestiges whose usefulness has waned. Several serious IEM diseases, such as Glut1 deficiency and phenylketonuria (PKU) result from mutations that prevent the relevant gene's expression in an active form. These two diseases, if detected before severe physiologic damage, can be managed nutritionally by limiting the availability of the substrate molecules handled by these proteins. Similar considerations apply to most complex organisms, especially homeotherms.

In fact, like PKU and GLUT1, recessively inherited loss-of-function mutations in enzymes and transporters constitute the bulk of IEMs. IEMs and most other results of mutation events are classified as “diseases” because they decrease the probability of the carrier of the mutation successfully reproducing. These mutations in germ line cells will face elimination unless the defect is addressed by the organism's metabolism in an alternative manner (e.g., a different pathway, a different environment).

While in most mitotic divisions our cells faithfully copy genetic material to replicate new cells, the process is not perfect. As part of the probability equation relating to the chemical interactions, in replicating billions and billions of new cells, genetic material copying is very, very, slightly unfaithful. In individuals, aging is correlated with an increasing load of mutated genomic material. Most mutations do not lead to cancer.

However, in rare but still a significant number of mutations, conditions exist to start a cell down a hyperproliferative pathway—that may, under a progressing set of conditions, eventually present as a cancer. The longer one survives, the more time there is for mutations to experience conditions favoring a route towards cancer.

This progression is quite relevant to cancer considerations. In cancer, a group of cells presents a group of mutations. But cancer itself is not naturally a strategy programmed in our genetic material. A specific group of cancer genes is not suddenly switched on. A series of events, genes switched on or off, pathways up-regulated, pathways down-regulated, nutrient uptake altered, etc., must all occur in the path to cancer.

In other words, cancer cells are living things and therefore follow chemical and physical laws and the principles of biology. Cancer itself is a complex disease. A cancer cell is not different in just a single respect from normal desirable cells. Many events are necessary to develop all the changes that make a cell cancerous. These events create conditions whereby the cells grow uncontrolled and thus must present with upgraded metabolic rates. These metabolic markers are targetable using, for example, biologic tools to recognize and destroy the aberrant cells.

Not every mutation improves survivability. Many mutations result in a non-functioning gene that if other features cannot compensate adequately for will mean that that cell will not survive. So as part of evolution, biological systems have evolved machinery to preferentially take out poorly functioning cells. One important process in this regard is called “apoptosis”. Apoptosis is a process that has evolved to remove undesirable cells. For example, apoptosis is triggered to remove cells at the base of baby teeth to facilitate disposal when adult teeth are coming in. Apoptosis also often selectively removes cells at times of stress. For example, several cells may be sacrificed during lean times to preserve nutrition for remaining cells. Cells that misfunction for one reason or another, for example, the membranes may become leaky to Ca⁺⁺ or intracellular structures or organelles such as cytoskeleton or mitochondria may present with compromised functions, will show abnormalities in their extracellular support functions. Many of these abnormalities increase probability of cell death through apoptosis.

But occasional mutations survive in some cells. Within the body, each cell, though guided by evolution, tries to survive. So, several mutations are expected to build up over a lifetime. As the cells continue to operate, many of the cells will harbor mutations. Some mutations may be silent; some mutations may be quiescent (not turned on, but available if stimulated). But all will be passed on when this cell divides. So what makes a cancer cell?

Cancer cells have been altered or have altered themselves to follow a metabolic program to enhance necessary biosynthesis and support that cell's and its progenies' proliferation. The changes may not be in the best interests of the organism. But concomitant with these metabolic changes must be changes that evade the organism's control of inappropriately behaving cells and that evade the apoptotic cell death protocols that evolution has provided in each cell's genetic instruction set.

One notable change in rapidly proliferating cells in general, but in cancer cells in particular, is a metabolic switch from using the mitochondria for efficient production of adenosine triphosphate (ATP) to favor a different, less efficient pathway for ATP production. This production process is carried out in the cytoplasm and produces less ATP per glucose molecule, and also ends with lactate, a three carbon molecule, instead of the single carbon molecule, CO₂. The metabolite, lactate, is a chemically energetic molecule whose energy is lost to the cell when the lactate is excreted using a slow but effective transport protein, monocarboxylate transporter protein (usually MCT4 or some MCT1). Lactate can be recycled by other organs in the body, e.g., the liver, to salvage the energy and carbon building capacities of the lactate molecule.

As mitochondrial ATP production is de-emphasized, cytoplasmic pathways using enzymes evolved for ATP production pathways become more active. Generally in pathway activation, expression is accentuated for the newly needed enzymes and transport proteins. Activation often starts at the transcription level which progresses through messenger RNA to ribosomal synthesis of extra copies of the proteins necessary for the pathways.

Some proteins are up-regulated. Others are down-regulated. Many will feedback through the pathway or regulate activity of other functions or cell proteins. For example, pyruvate kinase M2 (PKM2) plays a part in the altered glucose metabolism characteristic of cancer. Inhibiting one or more such enzymes using a virus, a small molecule or biological inhibitor and/or ligand starvation or product feedback negative feedback may be used with the systems and methods of this invention.

When pyruvate kinase M2 (PKM2) interacts with phosphotyrosine-containing proteins, it inhibits their enzyme activities resulting in an increased availability of glycolytic metabolites the cell then uses to support and encourage cell proliferation. An alternate, pyruvate kinase M1 (PKM1), same gene but processed differently (alternative splicing) within the cell, does not share this outcome. It can therefore be said that favoring genetic processing conditions that increase PKM2 at the expense of PKM1 is one factor supporting cancer development. While a mutation in the pyruvate kinase gene itself may affect splicing, a mutation in another gene or even an extracellular signal turning on or accentuating another path within the cell may be part of this cell's path to cancer.

As cells collect mutations, many will be culled by the organism's defenses which recognize damaged/unproductive cells. But occasionally a cell presenting a mutation leading towards a cancerous cell metabolism will evade these defenses and continue to reproduce. Several of the reproduced cells may be additionally mutated with each division. The same stress that may have encouraged the premiere mutation may encourage subsequent mutations and/or the premiere (or a subsequent) mutation may provide added stress encouraging still more mutations. Often cancer cells will carry a mutation that interferes with recognizing and repairing gene copying mismatches. Many of these mutations may still be removed by the organism's survival processes, but in rare, but significant to the organism, occasions multiple mutations can increase survivability of that cell line and continue to proliferate with continuously expanding mutations carried in the cell line's genome. At some point the collection of mutations and resultant metabolic responses will be sufficient to escape organismal control and will favor proliferation over the function the organism would like that cell type to perform. Many abnormalities can underlie the different cancers, but they each result in a common outcome. Regardless of specific initiating event(s) cancers all share the trait of improperly controlled hyperproliferation.

### SUMMARY

Cancer cells present as a disease characterized by an undesired expression of numerous traits, particularly traits leading to a rapid cell division. A cell's life can be defined as the sum of all its chemical reactions. Since cancer cells differ from normal cells, their chemical reactions (aka metabolism) must, by definition, also differ.

Cancer cells arise from diverse tissues and from many, many differentiated cell types, but at the root of all cancers is that cell's increased rate of making new cells, that is: hyperpro-liferation. Every time a cell proliferates it splits to create two cells—each of which requiring its own membrane, cytoskeleton, nucleus, mitochondria and other organelles. This duplication requires the cell to accelerate synthetic pathways and several additional pathways that support accelerated synthesis. The resulting two cells will require a doubling of DNA for duplicated nuclei, additional membrane lipids and proteins to cover the increased surface:volume ratio, extra endoplasmic reticulum, golgi, mitochondria, lysosomes, etc. to be split between two cells during mitosis. Mitosis itself is a resource hungry process requiring a slew of catabolic and anabolic events. In essence, a metabolic push is necessary to provide an additional set of all cellular components and the temporary resources and energy necessary to divide the cell into two. This accentuated metabolism can be employed to guide intercourse between i) an involved party, e.g., an anti-cancer compound, a probe, or other therapy, and ii) the cancerous, i.e., metabolically modulated cell(s). In this invention the anticancer probe comprises a biologic organism adapted to bind to and infiltrate into the hyperactive cancer cell.

Regardless of the cell type originating the cancer, all cancer cells will present this increased uptake of nutrient building blocks into the cell and increased use of the nutrients (reactants) in various chemical reactions to make necessarily increased products. The products will include products useful for sustaining the cell and by-products such as waste chemicals and heat. While there are some common chemical waste products of metabolism, one ubiquitous product (since in general metabolism is exothermic) is an increased heat output.

Since cancer cells produce more heat than surrounding cells, increased temperature is a metabolism specific, local, let's say, “nanomarker”, that can be used to identify and target these hyperactive cells for their destruction. While not an essential marker for all means of attacking cancer metabolism, heat can serve as a back-up confirmation or trigger signal for turning on natural innate and adaptive immunities and/or for making available one or more anti-cancer system(s) and method(s) in the identified cells.

The metabolic shift underlying increased metabolism deemphasizes the production of ATP through the electron transport chain (ETC). Pyruvate is not fed into mitochondrial metabolism, but rater is converted to lactate and transported chiefly by monocarboxylate transporter 4 (MCT4) wherethrough H⁺ and lactate are delivered to the cell's exterior space. The H⁺ thus transported results in a decreased pH that coexists with the increased temperature.

### BRIEF DESCRIPTION

Cancer cells are differentiated by their altered and increased metabolisms. The altered metabolisms can serve as identifying markers, targeting markers and/or markers that signal or trigger a therapeutic intervention. The unbalanced metabolism can be used as an important marker identifying the altered cells.

The identification, targeting and triggering can include mechanics that are very high tech. For example, physical or electronic nanoparticles can be configured with nanosensor capabilities. These man-made tools represent newer components that we have recently learned to make at a cellular and sub-cellular scale. However, developing life has spent eons designing molecules, microscopic and macroscopic organisms to maintain and control life. These life's tools can be adapted advantageously to repair and correct developmental and metabolic errors such as proliferating cancers.

Less technological applications of the invention are also available. Chemicals, especially lipid compositions, are heat responsive. Following the activation energy theories involved in completing a chemical reaction, including those facilitated by catalytic enzymes, all chemical reactions are temperature dependent. Thermo-dependence is even more evident in enzymatic reactions where subtle temperature changes can induce profound changes in a protein's or RNA's folding and activity. According to these three-dimensional models, a complex molecule's binding site(s)require stability in the interactions of multiple hydrophobic and hydrophilic parts of a molecule. At a low energy state (lower temperatures) the molecule's kinetic energies will be insufficient to dislodge hydrophobic and e.g., hydrogen bonds that maintain a three dimensional shape conducive to the catalyst presenting a ligand's reactive site(s) to another reactant. Increased temperature can increase random kinesthesis in the molecule and disrupt the appropriate three-dimensional configuration. In the membrane, interactions between lipids changes with temperature as the constituents in the bilayer present with a more solid or more melted form. The melted state of the membrane or a portion thereof (e.g., disordered or raft portions) can govern its ability to meld with other lipids or present integral membrane proteins.

Molecular biologists have several decades experience using temperature to change nucleic acid, folding, binding and activity and are adept at engineering sequences to fold or unfold at desired temperatures. Nucleic acids can be engineered to produce a protein of interest, including proteins whose range of temperatures where they are active is an engineering consideration, using available and improving software. Nucleic acids whose transcription, processing or translation is required to make the proteins can also be engineered for desired temperature dependence. Protein shape is determined by its primary sequence of amino acids. But this sequence folds and holds shape dependent on associative proteins, ligands in a binding or modifier site, temperature, hydrogen binding, salt, ionic strength, etc. Such proteins with temperature, salt, pH, etc., sensitivities can be incorporated into lipid membranes and/or their nucleic acid based instructions are tools frequently applied by microbiologists for modifying molecules, cells or organisms.

Experienced biologists, engineers, chemists, etc., now have available technology including hardware, software, artificial intelligence, etc., that allows close approximation in silico of protein foldings, temperature, pH, lipid, osmotic, ionic pressure, ionic strength factors and how these affect relevant components, for example, a specifically designed or selected lipid mix may intercourse and blend into another, such as a virus and vesicle, virus and membrane, vesicle and protein, vesicle and membrane, etc.

Another important feature common to the metabolic shift of cancer cells is the decreased reliance on the mitochondrial ETC for making high energy phosphates, e.g., adenosine triphosphate (ATP). To make the ATP that is required in amplified amounts to support the increased metabolism that supports the hyperproliferation, cells switch metabolic paths to emphasize a glycosylation process that ends with lactate⁽⁻⁾ and hydrogen ion (H⁺) as byproducts. The additional H⁺ ions depress the pH (a measurement indicative of H⁺ concentration). Another common byproduct is an increased abundance of various reactive oxygen species (ROS) such as H₂O₂ and .O₂⁻.

These chemical signatures can be used in addition to or as alternative to the heat signature given off by cancer cells for identification and targeting. The local pH can also be used as an activator or triggering mechanism extracellularly and/or intracellularly. Reactivity of molecules changes with protonation status which is dependent on pH. ROS species are very reactive and therefore will have greater applicability as an intracellular activator, but in specific circumstances these can be used as an activator signal or as a switch signal to be amplified in an extracellular application.

Although not observed in every cancer cell type, the increased metabolism results in a modified plasma membrane. Some modifications are for stability, such as slightly longer fat chains in the membrane to raise the lipid melting point to coordinate with the increased heat of metabolism. Most cells also have increased numbers of membrane transporters, e.g., to facilitate nutrient uptake and waste disposal; some cancer cells express binding or transport proteins not normally expressed in the neighboring more properly differentiated cells. In other instances, a transporter is found at extremely elevated concentrations in the membrane to support the substantially increased needs to transport some raw nutrients, such as amino acids and/or glucose. While these may be available as secondary targeting or trigger mechanisms, the primary mechanism—increased need for certain chemical reactions within the hyperproliferating cell—is a fundamental mechanism underpinning the identifying, targeting mechanisms of this invention.

Any available targeting or delivery means known in the art can be used including synthetic particles or devices, viruses, bacteria, etc. For example, a virus, e.g., a DNA or RNA virus can be engineered to deliver a therapy to the target cell's interior. The targeted cell will be a part of an organism, but the target may be selectively distributed, for example through injection, infusion, nano-particle delivery, etc. the delivery may be systemic or focused, e.g., to a specific part or parts such as a region, a tissue, an organ, etc.

In the example of a reovirus, the activated ras oncogene renders the cell more prone to infection by a virus since the activated Ras system deactivates a cell's antiviral defenses. Such an engineered retrovirus or other vector know in the art is therefore a viable courier for a variety of therapeutic strategies to modulate intracellular metabolism. A phase I/II study of intravenous reovirus in patients with melanoma (MAYO-MC0672 (NCI trial)), which has been performed. In this study, patients received systemic administration of reovirus at a dose of 3×10¹⁰ TCID₅₀ per day on Days 1-5 of each 28-day cycle, for up to 12 cycles of treatment.

Other cancers of interest for reoviral therapy include: pericutaneous tumors, prostate cancer, glioma, metastatic ovarian tumors, head and neck tumors, metastatic sarcomas, non-small-cell lung cancer, squamous cell carcinoma lung cancer, pancreatic cancer, fallopian tube cancer, metastatic melanoma, colorectal cancer, etc. These studies investigated reoviral advantageous infection of cells compromised by ras activation.

Vesicles, for example, liposomes, are another alternative whose membranes can be engineered to be sensitive to heat, pH, ROS or other chemical attractant or binding agent. Nanoparticles, including specifically designed nanosensor-particles can also be employed as couriers. Viral particles may be kept under conditions to interchange their lipid content with vesicles to change envelope fluidity and alter their selective merging with membranes they may encounter. Bacteria, especially bacteria that operate as intracellular parasites, represent organisms that can be adapted for desired extracellular and intracellular activities then applied therapeutically to recognize and eliminate cancerous cells. And non-biologic sensors, e.g., nanochips, may be delivered to the cytoplasm by being inserted into a viral envelope to take advantage of the abilities viruses have developed to enter and infect cells.

These adapted organisms and analogous nanoparticles can be supplied in the vicinity of a tumor or may be applied more systemically, such as in blood or lymph vessels. One species of nanoparticle we can make has a form of nano-motor, or means of moving itself like flagellate cells. These can be random or can be configured to be thermotaxic (move towards or away from a heat source) or chemotaxic (move along a chemical gradient, such as a pH gradient). Phototaxic (responsive to light—electromagnetic radiation, radio waves) sensors are another example, but these would be effective only close to the skin using ambient light or as secondary sensors responsive to a primary sensor that directs the secondary sensor to act at an identified location. Nanoparticles can also be configured as receivers of electromagnetic radiation. Nanoparticles compartmentalized for example by physical and/or chemical means can be queried to confirm location and if desired about the particle's surroundings. For example, the particle may report back an indication of temperature, pH, and/or other parameter programmed into the sensor. When the sensor is configured as an antenna, electromagnetic energy can be transmitted and converted to heat energy at the target location. While not essential for this invention, sophisticated nanoparticles, might be used to deliver and monitor delivery of, for example, bioparticles like viruses or bacteria.

While technology may be the source of many nanomarkers in the press, naturally occurring events that produce a detectible signal when at the biologic or macromolecular scale in a sense these may also be termed as nanomarkers.

As mentioned above, a sensor nanoparticle may also be a reporter nanoparticle, a courier nanoparticle and/or a signal nanoparticle able to deliver a preprogrammed substance or to recruit other couriers for delivery when a preprogrammed event is reported. Nanoparticles can be mostly physical in their action, may include chemical elements to aid in sensing or for delivery and may even transport biologic cargo(es) depending on the whims of the nanoparticles creator(s).

An intriguing application of nanoparticle chemistry involves introducing seed particles with one portion having high affinity for a ligand of interest, for example, a membrane receptor, a metabolite, a specific nucleic acid. Nanoparticles can grow the seed to form a larger molecule, perhaps a stronger antenna, perhaps a stronger antigen for recruiting immuno-defenses of the organism, perhaps disabling nucleic acids and causing havoc in the vicinity. Necrotic or apoptotic death may be the desired response. Nanoparticles can self-direct movement along a chemical or biological gradient and when concentrated at a gradient maximum act as nano-identifiers. For example, an enzyme may be activated by a chemo-attractant, e.g., H⁺, or a larger substrate, agonist, antagonist or cofactor, thereby providing a motive force in the direction of the higher concentration that is greater than the motive force where the concentration is lower. Other examples include conscription of “walking” enzymes (picture a polymerase like DNA or RNA polymerase or ribosomal polymerases) that can transport a cargo as they move along a gradient. A switch mechanism, such as sensitivity to a physical or electromagnetic frequency can transform these nano-identifiers into targeting and delivering devices. As an alternative they may serve as primary identifiers and targeters serving as a nanomarker for a secondary triggered anticancer response. Non-covalent binding, e.g., hydrogen binding, reversible or equilibrium binding such as protonation, etc., or temporary or permanent modification such as hydroxylation, oxidation-reduction, phosphorylation, etc., may serve as a signal or active modulator for some embodiments of the invention. A virus or bacterium could be configured to interact with such nanoparticles.

As an alternative application of nano-technology to biology, nano structures can be used to connect two distinct sites. For example, nano-tube structures can be made to conduct electricity or light between the site of interest and another device, perhaps outside the organism. Many configurations using nano-tube structures are available including, but not limited to, for example: i.) The nano-tube may transmit information interacting between a sensor and receiver. ii.) The nano-tube may act as a courier for small molecules or biomolecules. iii.) A photo-activation signal can be transmitted through fiber-optic nano-tubes. iv.) Electrical pulses can be transmitted through conductive nano-tubes. v.) Salts and/or nutrients may be precisely delivered. vi.) Plasmids, phages, small bacteria, virus particles may be delivered to a precisely known site. Nanotubes for biological applications have been synthesized as carbon nanotubes. Membrane based (lipid bilayer) nanotubules projecting from one cell to another have been used for transporting cytoplasmic content, including structures as large as mitochondria, from one cell to another.

Synthetic nano-tubes can be nano-surgically manipulated using micro-robotic signaling to desired locations and effectors within or at the ends of such nano-tubes can react automatically to predetermined stimuli such as pH thresholds, enzymes or enzymatic substrates, and/or temperature. Reaction may involve turning on, e.g., an electronic, biochemical, physical or chemical signal to attract and/or induce biomarkers or events; and/or a signal effective at the site to modify the surrounding cells' behaviors.

Recognizing that the plasma membrane is a lipid bilayer and has a mosaic of proteins, glycolipids, lipoproteins, sterols, glycoproteins, etc., the fluid mosaic membrane lipid bilayer model popularized in the 1970s has been updated to include a conceptual structure referred to as “lipid rafts”. Lipid rafts are believed to exist as constantly changing structural components floating in plasma membranes. Lipid rafts are believed to play an important role in many biological processes, especially signal transduction, apoptosis, cell adhesion and protein orientation and sorting. Membrane proteins and lipidated peptides, carbohydrates or proteins either reside in, form the boundary of or may be excluded from such rafts, depending on the molecule's physical/chemical properties. Since membrane binding and transport of molecules and signals across the cell membrane is the means through which cells interact with their environment including neighboring cells, lipid rafts are understood to play critical roles in many biological processes including viral infections.

The plasma membranes of eukaryotic cells comprise literally hundreds of different lipid species. The bilayer has evolved the propensity to segregate constituents laterally. This segregation arises from dynamic liquid-liquid immiscibility and underlies the raft concept of membrane subcompartmentalization. Eukaryotic membrane lipids are mostly glycerophospho-lipids, sphingolipids, and sterols. Mammalian cell membranes predominately comprise but one sterol, namely cholesterol, but the membrane comprises several hundred of different lipid species of glycerophospholipids and sphingolipids. In glycerophospholipids the head group of varies, also the bonds linking the hydrocarbon chains to glycerol, and the length and location and degree of saturation fatty acids provide distinguishing molecular features including how they sort amongst each other. Similarly, sphingolipids have the combinatorial propensity to create diversity by different ceramide backbones and, above all, at least 500 different carbohydrate structures at the head groups of the glycosphingolipids. Cholesterol interacts preferentially, although not exclusively, with sphingolipids due to their similar carbon chain structure and the saturation of the hydrocarbon chains. Although not all of the phospholipids within the raft are fully saturated, the hydrophobic chains of the lipids contained in the rafts are more saturated and tightly packed than the surrounding bilayer. Cholesterol then partitions preferentially into the lipid rafts where acyl chains of the lipids tend to be more rigid and in a less fluid state. Cholesterol is the dynamic “glue” that holds the raft together.

Molecule for molecule, cholesterol is often close to half the cell membrane molecules. But, since it is smaller and weighs less than other molecules in the cell membrane, it makes up a lesser proportion of the cell membrane's mass, generally ^{˜}20%. Cholesterol is also found in membranes of cell organelles, where it usually makes up a smaller but still significant proportion of the membrane. For example, the endoplasmic reticulum, which is involved in making and modifying proteins, is but 6% cholesterol by mass and the mitochondria, comprise about 3% cholesterol by mass. Similar to cells and their organelles, viruses and bacteria also comprise lipid constituents.

Given the role of mitochondria in oxygen consumption, metabolism and cell death regulation, alterations in mitochondrial function or dysregulation of cell death pathways contribute to the genesis and progression of cancer. Cancer cells exhibit an array of metabolic transformations induced by mutations leading to gain-of-function of oncogenes and loss-of-function of tumor suppressor genes that include increased glucose consumption, reduced mitochondrial respiration, increased reactive oxygen species generation and cell death resistance, all of which ensure cancer progression. Cholesterol metabolism is disturbed in cancer cells and supports uncontrolled cell growth. In particular, the accumulation of cholesterol in mitochondria emerges as a molecular component that orchestrates some of these metabolic alterations in cancer cells by impairing mitochondrial function. As a consequence, mitochondrial cholesterol loading in cancer cells may contribute, in part, to the Warburg effect stimulating aerobic glycolysis to meet the energetic demand of proliferating cells, while protecting cancer cells against mitochondrial apoptosis due to changes in mitochondrial membrane dynamics. The presence/absence of cholesterol regulates fluidity, which is the reason why the contents of cholesterol and other lipids are critical cellular and organelle structural components. Membrane dynamic processes involve biophysical concerns relating to fluidity which is controlled by lipid content and proteins in and on the membrane. Mitochondrial fusion/fission balance is critical to maintenance of proper cell functions. Altered fluidity can upset the balance and therefore the cell's energetic machinery.

Below the melting temperature (Tm), the membrane is gel like. The presence of cholesterol prevents ordered packing of lipids, thus increasing their freedom of motion, or in other words increasing membrane fluidity. Above this Tm (dependent on lipid content, especially cholesterol), the membranes are in liquid disordered state, the rigidity of cholesterol ring reduces the freedom of motion of acyl chains (trans conformation tends to increase order and help define the rafts. The decreased fluidity and higher order allows for a stronger resistance to disrupting influences such as polar molecules and thus decreases permeabilities to especially foreign substances such as water and nitrogen and oxygen containing compounds.

Without cholesterol, cell membranes would be too fluid, not firm enough, and too permeable to many molecules. Because the fatty acids are longer and more saturated (straighter), they aggregate more, which cholesterol also helps. That ordered part of the membrane is also thicker, making it better suited for accommodating certain proteins. Since the fatty acids in lipid rafts are longer, raft phospholipids move in sync with the phospholipids on the opposite side of the membrane. In the disordered portions of the membrane, the phospholipids on one side of the membrane move independently of those on the other. By stabilizing certain proteins together in lipid rafts, cholesterol is important to helping these proteins maintain their function.

Lipids, e.g., glycolipids such as a glycerolipid that has one fully saturated chain and one partially unsaturated chain could function as a surface-active component, a hybrid lipid or a linactant. These linactants would lower the line tension between domains by occupying the interface, with the saturated anchor preferring the ordered raft and the unsaturated fatty acid interacting with the less ordered lipid environment. Small finite-sized assemblies of disordered and ordered lipid domains separated and stabilized by these hybrid lipids could be expected to form as equilibrium structures. In the viral envelope especially but even in the plasma membrane with its generally lower protein content proteins, especially with multiple transmembrane domains such proteins should also act as linactants. Several protein structures would be ideally suited for this purpose. For instance, proteins that have both a GPI anchor and a trans-membrane domain have been identified, in which the GPI anchor could be raft-associated with the trans-membrane domain facing the non-raft bilayer. Another such protein is the influenza virus M2 protein, which seems to occupy the perimeter of the raft domain that forms when the virus buds from the plasma membrane. N-Ras has also been proposed to act as a linactant in the cytosolic leaflet of a raft.

Many studies indicated that membrane rafts must play an important role in the process of virus infection cycle and virus-associated diseases. Many viral components or virus receptors are exclusive to or concentrated in the lipid raft ordered microdomains.

Viruses have been divided into four main classes, non-enveloped RNA virus, enveloped RNA virus, nonenveloped DNA virus, and enveloped DNA virus. General virus infection cycle is also classified into two sections, the early stage (entry) and the late stage (assembly and budding of virion).

Several studies have demonstrated the localization of viral structural proteins in membrane rafts and the effects of raft-disrupting agents (mainly removing reagents and synthesis inhibitors of cholesterol) in the replication processes of several viruses, including retroviruses (Retroviridae), RNA viruses (classified into Picornaviridae, Caliciviridae, Astroviridae, Reoviridae, Flaviviridae, Togaviridae, Bunyaviridae, Coronaviridae, Rhabdoviridae, Arenaviridae, Filoviridae, Orthomyxoviridae, and Paramyxoviridae), and DNA viruses (classified into Parvoviridae, Papovaviridae, Adenoviridae, Herpesviridae, Hepadnaviridae, and Poxviridae).

Initial viral infection arises via endocytosis or by injection of viral proteins and genes directly into the cytoplasm, by fusion of the viral envelope or by destruction of the viral capsids. Transcription and replication of DNA viruses except poxviruses generally happens inside the nucleus, whereas those of RNA viruses occur in the cytoplasm. However, influenza viruses are exceptional as RNA viruses with at least a major genome duplication occurring after transport to the target host cell nucleus. Before, after and during the transport and duplication processes, the innate immunity of the cell can act on the viral proteins and vRNA.

Once the progeny viral components have been produced, fragments are transferred to some organelles or to the plasma membrane, where formation of the progeny virus is processed by assembly and/or budding. Based on the viral outer boundary structure, virus particles are classified into enveloped viruses (Herpesviridae, Hepadnaviridae, Poxviridae, Flaviviridae, Togaviridae, Retroviridae, Bunyaviridae, Coronaviridae, Rhabdoviridae, Arenaviridae, Filoviridae, Orthomyxoviridae, and Paramyxoviridae) and nonenve/oped viruses (Parvoviridae, Papovaviridae, Adenoviridae, Picornaviridae, Caliciviridae, Astroviridae, and Reoviridae).

The envelope of virus particles is acquired from the plasma membrane of the cell surface, Golgi apparatus, and/or endoplasmic reticulum (ER) by budding off these membranes. Influenza viruses, which are highly transmittable pathogens of severe acute respiratory symptoms in various animals including many macroorganisms, especially mammals and birds, internalize into host cells through multiple pathways including clathrin-independent and caveola-independent endocytosis after binding of the virus to a terminal sialic acid linked to glycoconjugates on the cell surface via viral surface glycoprotein, hemagglutinin. After transportation of the virus to late endosomes, low-pH-dependent conformation change of hemagglutinin induces membrane fusion of the viral envelope with the endosomal membrane. Then viral ribonucleoprotein complexes (RNP) including the viral genome are released to the cytoplasm of host cells by proton influx of viral ion channel M2 protein that requires binding with cholesterol. Influenza virus particles consist of the viral RNP with an envelope that includes two spike glycoproteins, hemagglutinin and neuraminidase (NA), and ion channel M2 protein on the outer surface and internal M1 protein and nonstructural NS2 protein on the inner surface. Membrane rafts are associated with the transmembrane domains and cytoplasmic tails of hemagglutinin and NA, with the short transmembrane domains of M2, and with NP but not with M1. Domains of hemagglutinin and M2 contain palmitoylated cysteine residues that can associate with lipids and cholesterol in rafts. Although these domains of NA are essential for the association with rafts, there is no evidence that NA possesses palmitoylated residues.

During the budding of enveloped viruses from the plasma membrane, the lipids are not randomly incorporated into the envelope, but virions seem to have a lipid composition different from the bulk host membrane. The virion envelope appears to be determined both by the virion protein content helping to order and select to surrounding lipids AND the presence and availability of specific lipids from which to extract. Although the cell and the culture conditions in which the progeny viruses are produced is a significant factor in determining lipid content and, to an extent, its Tm, the proteins present or deleted from the virus and mutations on these proteins as well as cell membrane lipid content and cell membrane proteins assisting in ordering the raft portions of the membrane are important factors.

Since the virus must contact the target cell before infecting it, recognizable features are used by viruses to attach to and gain entry into their targeted cell. Any surface feature including, but not limited to: a membrane protein, a weldable lipid blend, a specialized raft, a glycoprotein, a glycoprotein, and/or any portion or fragment thereof, etc., might be recognized by a targeting virus. Viruses may be engineered using molecular biology and/or mutated or adapted using for example serial culture to obtain viruses that recognize one or more selective feature.

All animal viruses must traverse cells' plasma membranes for access to the cells machinery to propagate the virus. Cell entry occurs by membrane fusion (in enveloped viruses). Although a protein rich capsid represents the outermost structure of naked viruses, it is surrounded by a targeted host cell-derived membrane in the case of enveloped viruses. Virus replication is a multi-stage process inside the respective host cell before viruses release to the environment to infect additional cells. Accordingly, to act as infectious agents viruses must cross the host cell boundary at least twice during a replication cycle, once when entering and once for exit and distribution.

In enveloped viruses, entry occurs by fusion of the incoming virus with, and cell lysis during budding of the nascent virus across a cellular membrane. Virus entry is specific for susceptible host cells and depends on the viral surface proteins and receptors exposed on the target host cell membrane. Most cellular receptors are surface proteins of various functions, but sugars (i.e., one of the sialic acids for influenza virus) and lipids can function as receptors. Virus entry is often enhanced by nonspecific binding, thus increasing viral residence times at the cell surface. This non-specific binding is often achieved by glycosaminoglycans (e.g., heparan sulfate), which promotes cell attachment of many different viruses by ionic or electric charge interactions. Simple plasma membrane fusion between envelope and cell appears to occur rarely—if it ever happens—and even viruses that can possibly fuse at the plasma membrane appear to commonly take an endosomal route. It appears that viruses rely on lipid rafts for entry. For example it has been shown that several non-enveloped viruses go through a raft-dependent entry pathway that requires cholesterol. Lipid rafts are also involved in enveloped virus entry as can be inferred from preferred binding to raft-associated viral receptors (e.g., GPI-anchored or raft-associated trans-membrane receptors). Enveloped viruses also present with a requirement for entry based on raft integrity and cholesterol.

The “endosomal sorting complex required for transport” (ESCRT) components found in cell and organelle membranes clearly play an important role in viral infection, especially in the release of many, but certainly not all enveloped viruses, important exceptions being the herpesvirus cytomegalovirus, influenza virus, and respiratory syncytial virus. These viruses recruit alternative cellular machinery or may employ viral proteins facilitating membrane scission. For example, the influenza virus M2 protein comprises an amphipathic helix that is both necessary and sufficient for vesiculation in vitro and generally for influenza virus budding in tissue culture.

Influenza M2 is a trans-membrane protein that self-associates to become a homotetramer providing proton-selective (H⁺) ion channel activity through the virion membrane. M2 binds to low cholesterol (lipid disordered) membrane regions to induce a positive curvature. M2 preferentially sorts to the phase boundary of phase-separated vesicles causing extrusion of the lipid ordered (lo) domain, dependent on the presence of the amphipathic helix. The M2 pore localizes to the neck of influenza virus buds in virus-producing cells. Mutation of its amphipathic helix causes late budding arrest similar to late domain mutations in other enveloped viruses. Apparently, M2 serves an analogous function as the ESCRT-III/Vps4 complex in other viruses. The influenza virus membrane is enriched in cholesterol and is more lo than the surrounding plasma membrane thereby creating line tension at the phase boundary demarcating the viral bud. M2 preferentially sorts at this phase boundary and apparently modulates line tension through lipid membrane interaction of its amphipathic helices.

As alluded to above, lipid rafts appear as subdomains of a cell's plasma membrane. The rafts comprise elevated concentrations of cholesterol and glycosphingolipids. They exist as distinct liquid-ordered regions of the membrane that are resistant to extraction with nonionic detergents. Lipid rafts generally contain 3 to 5-fold the amount of cholesterol found in the surrounding bilayer. The lipid rafts are enriched in sphingolipids such as sphingomyelin, which is typically elevated by 50% in comparison to the disordered plasma membrane regions. As a result phosphatidylcholine levels are decreased leaving similar choline-containing lipid levels between the rafts and the surrounding plasma membrane.

Each raft is apparently small in size, but the many rafts constitute a relatively large area of each plasma membrane. To exist as a demarcated entity each raft must have a distinguishing protein and lipid composition different from the disordered lipid membrane through which it floats, but all rafts all rafts of a cell are not mandatorily identical in terms of either the proteins or the lipids that they contain.

Strains of vaccinia virus, herpes virus, vesicular stomatitis virus, senaca virus, Semliki Forest virus, ECHO or REGVIR virus, and monstrously attenuated polio virus have been similarly tested and characterized in cancer cells or in animals with cancers for their inherent cell killing effects primarily targeted at cancers. In retroviruses, the capsid is important throughout the life cycle. Quaternary arrangements in mature capsid cores are structurally conserved among retroviruses. Water and ions including H⁺ and Ca⁺⁺ control interactions at several interfaces in the mature capsids. Accordingly, successful viral infection is extremely dependent on the activity of water and the ions present in the environment surrounding and within the targeted cell. A paper published more than a century ago, before viruses were discovered noted that in some patients an infection with a flu like disease improved some patients who were afflicted with leukaemia. Dock, George: THE INFLUENCE OF COMPLICATING DISEASES UPON LEUKAEMIA. The American Journal of the Medical Sciences (1827-1924), Vol. 127(4), p.563. American Periodicals Series II April 1904. Whether this was an effect related to fever dehydration, compromised breathing, or other flu-like symptom(s) or events was not manifest. The identification and targeting using a virus can be engineered or selected to increase selectivity and efficacy. Bacteria when grown under selective conditions, for example available lipids, temperatures ionic strengths, etc. will see their membrane fluidities and reactivities similarly modified. The bacterial genome can also be manipulated for desired effects.

Owing to the strength and significance of hydrogen bonding in biomolecules, pH balance is extremely important to the biochemical reactions that define the gamut of metabolic processes—and cell and organism physiology as a result. As a rule, blood will be in a slightly alkaline range of about 7.35 to 7.45. Management of the pH is so important that the body's primary regulatory systems (especially breath, circulation, and renal controls) closely regulate the overall acid-alkaline balance and will counteract, on a system wide or whole organism basis, pH aberrations caused by local metabolic anomalies. The result is that the gross pH is generally maintained within a “normal” range irrespective of local stresses.

Certain viruses (including the rhinoviruses and coronaviruses that are most often responsible for the common cold and influenza viruses that produce flu) infect host cells by fusion with cellular membranes preferably modified by increased temperature and at low pH for optimal action in this invention. These are referenced as “pH-dependent viruses.” Viruses can exhibit similar temperature and/or pH sensitive selectivity through modification of the viral recognition proteins to bind, for example, an MCT4 or similar protein expressed on the surface of the metabolism-altered heat producing cells. Intracellularly active bacteria perform similar functions in recognizing and entering cells. The bacteria, as larger organisms may often prove more efficient at rapidly killing the cells they enter.

Drugs that increase intracellular pH (alkalinity within the cell) have been shown to decrease infectivity of pH-dependent viruses. Since such drugs can provoke negative side effects, the obvious question is whether more natural techniques can produce the same or an opposite result. A more direct approach using a simple biologic such as a bacterium may present less onerous side effects.

Influenza virus, a member of the family Orthomyxoviridae that is an enveloped virus containing a genome comprising eight segments of negative-sense single-stranded RNA (ssRNA) has strains that are especially sensitive to pH for their target cell binding and thus can be used to preferentially target cells in low pH environs produced by cancer cells that skew metabolism towards lactic acid as a metabolic product. Influenza is a lytic virus which rapidly kills the host cell when the offspring virus are released. Since flu is a lytic virus the host cell genome is immediately incapacitated and the cell can no longer divide to form offspring cancer cells. This contrasts with retro-viruses like herpes and HIV which follow a lysogenic cycle, inserting viral reverse transcribed DNA into the host genome while the host remains viable. When the host cell divides, the lysogenic phase retrovirus remains incorporated into both new cells' genomes. But eventually the viral DNA is activated to produce large quantities of new virus particles whereupon that host cell ruptures (is destroyed) as the new particles are released. However, copies of the viral genome remain dormant in the multiple divided cells with “sleeper” retro DNA. For this consideration, retrovirus is a stronger candidate for genetic engineering of cells to correct genetic flaws, while non-lysogenic viral infection is better suited for targeting and destroying invading, diseased or otherwise unwanted cells. Influenza A can swap one or more of its 8 RNA strands with co-infecting viral particles or may undergo a drift mutation, perhaps a single nucleotide base that changes a single amino acid or results in early truncation, with a robust change in transmissibility, infectivity ad/or mortality.

Hemagglutinin on the surface of the flu virus is instrumental for binding to and infecting target host cells. The 2009 human swine flu is particularly illustrative of this phenomenon. Hemagglutinin mutated to become more acid stable as this H1N1 virus shifted from swine to humans. This lowered the pH at which the flu hemagglutinin was activated. The activation process triggers an irreversible change in the hemagglutinin's shape that then fuses the virus and target cell. The pH of activation is known to vary amongst various flu viruses. Avian and swine viruses are generally activated at about pH 5.5-6.0 compared to a two-fold higher [H⁺] or pH about 5.0-5.5 predominant for human flu viruses. In the context of the 2009 pandemic, H1N1 swine viruses which were previously activated at pH 5.5-6.0 mutated to become activated at pH 5.5 at the pandemic inception and as the pandemic progressed, the activation pH of the H1N1 pandemic virus declined to 5.2-5.4. This mutation process can occur naturally as pH of the target changes or for purposes of the present invention culturing susceptible cells at decreasing pH levels, where targets may be selectively cultured to decrease their pH ranges for survival and growth or by switching the target cell line if preferred. Lowering the activation pH of the hemagglutinin may be one means of selectively targeting cells that favor a more acidic metabolism.

The infection process starts when hemagglutinin binds to a monosaccharide sialic acid present on the surface of the target host cell. Influenza viruses deliver their genomes into the nucleus as multiple single-stranded RNAs. Newly synthesized viral RNA will then exit the nucleus for assembly into virus particles on and with the plasma membrane. The viral envelope can thus be engineered by choosing the host cell used to manufacture the virus particle.

In the low-pH environment of the endosome, the hemagglutinin is activated by a conformational change triggering its membrane fusion activity. The viral membrane fuses with the limiting membrane of the endosome to release the nucleocapsid into the cytosol. Flu virus delivery of genetic material is rapid. The total infection period—from docking onto the cell's surface to the RNA entering the cell nucleus—is two hours. Influenza A because of its ability to mutate by both antigenic drift and shift is a preferred type of influenza virus for engineering select mutations in furthering this invention. In a low pH environment, the pH stabilized viral particle may facilitate development or may take advantage of tunneling nanotubes to pass infectious RNA to neighbor cells without necessity for forming an envelope.

The US Centers for Disease Control Recommends annual flu vaccination for everyone older than six months. This is because influenza virus is continuously changing its antigenicity. Small changes in tropism antigenicity, virulence, etc. are referred to as “drifts”. Antigenic drifts are small changes in the viral genome that only minorly impact antigenicity (the process of being recognized by our immune systems) in the short term, but over time may change enough so that increasing numbers of vaccinated people will no longer mount an adequate antigenic response. These drifts are especially prevalent in both types A and B influenza and are a major factor driving annual flu vaccine revisions.

Tropism, the preferred target cell of a virus also may undergo drifts. This tendency to drift, while inconvenient for long lasting vaccine effectiveness against the drifting organism can be applied for beneficial purposes using serial selection. By exposing the viral culture to higher and higher temperature cell membrane targets, the most prolific viruses will soon predominate the cultures. In a repeated (serial) fashion, viral selectivity for the characteristics of the viral receptor can be engineered, not by classical molecular biology, but by the general process used for selective breeding. The “engineer” modifying the virus does not have to predict which genetic alterations will result in which affinity modifications, the millions of random mutations can be selected for desired traits.

For example, a virus may be serially adapted to favor higher temperature membranes. A separate culture may be selected for improved affinity at lower pH.

Shifts, while not as precise due to entire strands being exchanged, can be especially relevant when multiple target traits are to be selected. Aliquots of different cultures with the desired characteristics placed in co-culture can be used to select for shifts optimally selected for the different traits. Such adaptive cultures are available tools for adapting rapidly growing biologics such as viruses and bacteria.

As an illustration, a class A influenza virus, e.g., H3N2, is cultured in a receptive host cell. (The H refers to the form of hemagglutinin; the N refers to the form of neuraminidase; human viruses have been H1, H2 and H3 and N1 and N2; H1N1 and H3N2 are most common infectious forms in humans. Similar distributions appear in domesticated animals that have been studied. About 20 hemagglutinins are known, while neuraminidases have been seen in over 100 varieties.) The pH is gradually decreased with subsequent passaging. Attenuation is monitored to assure the virus remains infectious to target cells other than the cultured cell strain. In a preferred embodiment attenuation is observed at normal pH, but infectivity remains at elevated [H⁺].

The temperature is also increased in culture to affect the content of the viral envelope to favor assimilation into membranes at increased temperatures. Alternatively, the low pH stable virus is allowed to mix with liposomes with higher melting temperature to transfer the liposomic constituents to the viral envelope lipid coating.

The resulting infectious virus is again screened or tested for selective infection at depressed pH and elevated temperature. Such virus may be delivered to a patient as a treatment for cancer, to target hyperproliferating cells and/or as a prophylactic event to seek out and eliminate cancerous cells that have not yet been outwardly observed, such as being palpated as a tumor mass.

Although H1, H2 and H3, and N1 and N2 are the common infecting hemagglutinins and neuraminidases respectively, others may mutate to be compatible with animal cells as hosts and able to cause disease and death. For example, the recent outbreak of bird flu was H7N9 killing several dozens of humans, but apparently was not able to replicate in a form transmissible from human to human. In another example, such virus with lytic potential but lacking transmission between untreated humans in contact with the recipient is prepared as a pH and heat targeting lytic vector. Influenzas B and C may be cultured and applied in the invention for similar considerations. An influenza A H5N1 virus, another drifted avian virus though weakly transmissible to humans, apparently requiring thousands of copies to infect a human can be extremely pathogenic as it may occasionally drift. Humans are merely the most studied influenza victims and thus are used herein for exemplary purposes.

Influenza A viruses are especially capable of inducing the expression of cytokine and proapoptotic genes in infected cells. Pathogenicity, cell lethality, replication efficiency, and transmissibility of influenza viruses depend on both viral genetic and host factors. Hemagglutinin protein binds receptors and mediates viral-cellular membrane fusion during viral entry is the primary antigenic target during infection. Hemagglutinin protein is a trimeric class I membrane fusion protein that sports in its ectodomain a membrane-proximal, metastable stalk domain that is capped with a membrane-distal receptor-binding domain. Hemagglutinin protein is readied for membrane fusion by cleavage of the hemagglutinin precursor into a fusion-capable hemagglutinin1-hemagglutinin2 complex. Some H5 and H7 hemagglutinin proteins can be cleaved by intracellular furin-like proteases to elicit systemic virus spread with enhanced virulence of such highly pathogenic avian influenza (HPAI) viruses.

Infection by influenza virus' hemagglutinin surface glycoprotein binds sialic acid-containing receptors on the plasma membrane of a target host cell. In general, H5N1 influenza virus hemagglutinin proteins bind preferentially to α(2,3)-linked sialosides. Whereas human-adapted influenza viruses bind preferentially α(2,6)-linked sialosides. A switch from α(2,3) receptor binding specificity to a(2,6) receptor binding specificity may be preferred in adapting avian influenza viruses for mammalian hosts.

Hemagglutinin proteins from different strains and subtypes vary in activation pH values with a range from ˜4.6 to ˜6.0. Hemagglutinin proteins from HPAI viruses normally exhibit an activation pH value at the higher end of the range ˜6.0, while seasonal viruses have lower pH activation values, ˜5.0 or less. H5N1 influenza virus isolates cluster in a range of ˜5.3 to ˜5.9. For individual viruses grown in sequential culture genetic drift is an effective tool for directed mutation towards a desired activation pH range to match that of a target host cell. For example, in H1, H3, and H7 influenza viruses, mutations that alter the hemagglutinin activation pH have been associated with changes in virulence in mice.

After receptor binding and internalization during influenza virus entry, the hemagglutinin protein is triggered by low pH to undergo irreversible conformational changes that mediate membrane fusion, and initiation of cell lethal infection either through apoptosis or other cell death or through lytic release of virus.

An initial stage of immunity occurs within the cell under attack by a foreign (pathogenic) genome. Pattern recognition receptors (PRRs) recognize pathogen-associated molecular patterns (PAMPs) on invaders to initiate both the near instantaneous intracellular innate and the delayed and lasting adaptive immune responses. Toll-like receptors (TLRs) comprise an important set of PRRs where TLR activation initiates induction of interferons (IFNs) and cytokines active in both innate and adaptive immunity. Humans have at least 10 TLRs (appropriately numbered TLRs 1-10). The various TLR proteins bind different type targets, for example, TLRs 1 and 2 are involved in bacterial infections through their recognition of lipopeptides (1) and lipopeptides, lipoproteins and glycolipids (2); TLR3 recognizes double stranded RNAs and thus is preferentially effective against viruses. TLRs 7,8 and 10 are activated in the presence of ssRNAs, especially of the types found in influenzas. TLR7 and TLR8 especially recognize GU of AU rich sequences of ssRNA viruses such as the Orthomyxoviridae family that includes influenza virus. TLRs are generally conserved across at least mammalian species.

Compared with seasonal influenza virus H1N1, highly pathogenic avian influenza virus H5N1 is a more potent inducer of TLR 10 expression. Influenza virus infection increases associated TLRs expressions which contribute to innate immunity through their sensing the viral infection. This leads to cytokine induction, especially proinflammatory cytokines and interferons. Since TLR 10 induction is more pronounced following infection with highly pathogenic avian influenza H5N1 virus compared with a less pathogenic H1N1 virus H5 influenzas are a preferred initiator of cell death.

However, although H1N1 viruses may be effective for infecting targeted animal cells, previous exposures to similar H/N epitopes may compromise access to target cells. Accordingly, it is advised to be cognizant of recent flu outbreaks that may have produced antibodies and other humoral reservoirs that might neutralize specific cell lines.

But as humans and other organisms have adapted to minimize and therefore better survive viral invasion, viruses also adapt to continue viral propagation. Among the 11 proteins encoded by influenza virus, the NS1 protein has been shown to block the production of IFN-β in infected cells. Such adaptations of an influenza virus allow it to evade host cell innate immunity.

For example, influenza viral protein NS1 serves to bind viral RNA with its RNA binding domain to shield it from contacting ssRNA sensitive TLRs and retinoic acid inducible gene-I (RIG-I) a protein recognizing dsRNA including looped ssRNAs that complementarily bind. When stimulated by binding RNA, the TLRs, RIG1 and the like induce type I interferon production. Some NS1 proteins also bind the tripartite motif-containing protein 25 (TRIM25) that works though the activation of RIG-I. NS1s apparently also can complex with RNA-dependent protein kinase (PKR) and inhibit it. Otherwise PKR is activated by binding double-stranded viral RNA and causes translation arrest in the cell nucleus including inhibition of viral protein synthesis. As another defense, the influenza virus M2 protein can inhibit P58IPK also inhibiting protein synthesis, and arresting host cell apoptosis.

Influenza PB1-F2 with a serine at position 66 is especially adept at inhibiting type I interferon production. This PB1-F2 binds to and inactivates mitochondrial antiviral signaling protein (MAVS). PB1-F2 protein is also associated with the induction of apoptosis and has a synergistic effect on the function of influenza virus polymerases PA and PB2. PB2 can also bind and inhibit the interferon promoter stimulator 1 (IPS-1) that normally promotes IFN-β production.

When to main desired function of the virus is to induce cell death in contrast to many previous uses of viral infection to deliver a gene for genetic therapy, defective viruses, i.e., viruses lacking a full component of genetic material and associated proteins to reproduce more virus particles, and/or notably non-virulent viruses, e.g., viruses easily attacked by the host cell innate immunity, can be considered as viable or even preferred embodiments for use in the present invention. For example, flu viruses with one, two, three, four, five, six, seven, or even all eight RNA strands absent or modified to be incapable of expression or to result in a strong anti-viral response and/or to lack viral defense against host cell antimicrobial defenses, even if no new viruses are made can still target the hyperproliferating cancer cell and by initiating cell apoptosis or other cell death, even non-productive lysis, serve the appropriate functions envisioned in this invention.

Adeno-associated virus (AAV) in contrast, cannot gain cytoplasmic access by membrane fusion. AAV comprises single stranded DNA (ssDNA). The AAV genome is manipulatable by inserting chosen genes, including genes encoding interfering RNA molecules. Various receptors can be selectively targeted. For example, secondary receptors including, but not limited to: fibroblast growth factor receptor (FGFR), 47/67 kDa laminin receptor, 37/67 kDa laminin receptor, hepatocyte growth factor receptor (HGFR), αVβ5 and α5β1 integrins, platelet-derived growth factor receptor (PDGFR), etc. have been observed as specific targets for binding by specific AAVs. Since AAV requires a helper virus in addition to a host cell for replication and packaging, viruses of this sort may be selected to avoid undesired contagion of the treatments, generally gene therapy constructs in recent literature. AAV has been mutated using site directed and random mutagenesis to alter its pH sensitive components.

Inside the cell the virus can reproduce to cause cell lysis or may induce suicide by the invaded cell as the ell attempts to sacrifice itself to avoid infecting other nearby cells of the organism. Pathogen recognition receptors (PRRs), including, but not limited to: RIG-I, MDA5, PKR (encoded by ISGs: IFIH1, DDX58, and EIF2AK2), react to accumulating vRNA and activate the cell's apoptotic pathways to direct the demise of influenza or other viral infected cells. Anti-apoptotic (Bcl-2, Bcl-xL, and Bcl-w) and pro-apoptotic (Bax, Bak, Bad, Bim, Bid, Puma, and Noxa) Bcl-2 proteins react to initiate the cascade of reactions leading to apoptosis with the early step involving mitochondria membrane permeabilization (MoMP) causing release of cytochrome c, apoptosome activation, ATP degradation, etc., ending with cell death. The concentration of vRNA is a critical result-limiting factor so apoptosis becomes more likely as viral RNA is synthesized. On the other hand, if the viral load were high enough, apoptosis could commence with virus entry even before the host makes additional copies of vRNA.

Mitochondrial participation in the cell death cycle following viral attack is apparent as PB1-F2 protein of influenza A viruses depositing on mitochondria where it interacts with VDAC1 and ANT3 to decrease mitochondrial membrane potential (MMP), which induces the release and self-associations of proapoptotic proteins that cause cell death. PB1-F2 also forms non-selective protein channel pores that lead to the changed mitochondrial morphology, dissipation of MMP, and efficient cell death. The M2 protein of influenza virus, another viroprotein that causes changed mitochondrial morphology and depletion of MMP, is another means whereby a viral infection can cause cell death.

For best efficiency when viral particles are used in a vectoring capacity, the couriers may preferably transport a molecule whose effects are multiplied in the cell, For example, the courier may carry RNAi with downstream effects on one or more pathways, may carry transcription factors, methylation factors, demethylation factors, an engineering cassette such as used in CRISPR/cas, a plasmid that can infect mitochondria, a ligand that opens a pore in an organelle such as the nuclear membrane or mitochondrial membrane, packets that increase expression of a protein or group of proteins to favor or disfavor one or more metabolic pathways, such as the Electron transport pathway of mitochondria, mitochondrial fusion or fission, anti-apoptotic or pro-apoptotic compounds such as Bcl or Bad, etc.

In addition to targeting the heat signature and decreased pH inherent in the lactate shift, targeting the MTC4 directly may be used for additional specificity and efficacy. The MCT4 being a transport protein has both intracellular and extracellular regions on respective sides of the plasma membrane. When an antibody is raised against MCT4 embedded in a membrane, Extracellular region specific anti-MCT4 antibodies have been raised. By incorporating Fab or antigen binding regions of such peptides onto a delivery instrument such as a liposome or engineered viral particle, delivery can be enhanced. For example, such antigen targeting fragment may be chimerized with a protein fragment of choice for embedding into the membrane of the delivery device. Recognition can be further improved by selecting binding fragments more strongly active in higher W and/or higher temperature environments.

Immune technology to purify and manipulate specific immune cell types with a goal of treating disease including specific cancers has evolved following President Nixon's declaration of war on cancer. Replacing or in conjunction with surgery, chemotherapy and radiotherapy, immunotherapy Genetically engineered immune cells have been investigated as treatment options for HIV and several cancers. Lymphokine-activated killer cells, cytokine-induced killer cells, and natural killer cells, can mediate cancer regression with non MHC restriction. Phase II/III trials using vaccine-induced expansion of tumor-specific effector T cells have shown promise.

In the absence of access to antibodies specific to a tumor's receptor molecule(s) such cell-based therapies though considered safe show limited efficacy. The present invention in immune-like targeting of MCP4 in conjunction with engineered elevated activities in higher temperature and lower pH environments (as made by cancer cells (and some pre-cancer cells) in general) Provides enhanced concentration and activity at the desired sites of action. T cell receptors (antigen-specific t cell proteins and tools are available from, for example, Astarte, Miltenyl, and other biomedical supply houses) and/or may be made and improved by one skilled in such art. The general approach avoiding the individualization of each recipients targeted cancer protein allows more rapid and less expensive therapeutic intervention.

An approach analogous to these strategies can be implemented with bacteria. Bacteria have an advantage over synthetic or viral based strategies in that the bacterial organisms have their own nucleic acid and polypeptide synthesis pathways. Once a bacterial strain is established, its asexual reproduction is accomplished as monoculture. Unlike viral vectors that require a host cell for reproduction and thus compromised culture conditions that must consider optimization of host cell growth and culture conditions as well as the viral optima, the bacterial monoculture can be optimized for maintaining the bacterial adaptations in a growth supportive medium. Several genus of bacteria have specialized to exist as intracellular parasites. When they eschew synthesizing bacterial transmembrane proteins, the bacteria avoid the cell based and humoral immunities of the organism.

Bacteria are generally easier to grow and maintain in culture than more complex cells, especially more complex cells that must support viral propagation. Facultative intracellular bacteria are a special class capable of self-propagation either intracellularly or extracellularly. For a manufacture phase, the extracellular propagation can be accomplished in a bacterial monoculture. Continuous asexual doublings can be accomplished and the monoculture harvested without the presence of the targeted cell. The culture can be optimized just to sustain bacterial characteristics and to support its growth without regard to needs of the more complex target cell. These bacteria, when properly selected in a manner analogous to the serial selection of viruses discussed above, can be used in a fashion analogous to the viral anti-cancer cell strategies, but are more efficiently and less expensively produced. Quality control can include analysis of the bacterial genome and/or periodic testing with a host target cell. Thus, facultative bacteria are one preferred feature capable of use in the present invention. Examples of such facultative bacteria include, but are not limited to: bartonella henselae, brucella, francisella tularensis, legionella, listeria monocytogenes, salmonella typhi, mycobacterium, nocardia, rhodococcus equi, yersinia, etc. When grown in culture, the facultative bacteria can continue to proliferate without intervention by a host organism's immune systems.

In special circumstances where the skilled artisan wishes to better control bacterial growth bacteria including, but not limited to: chlamydia, coxiella, rickettsia, etc., may be cultured with the understanding that they only proliferate inside a target cell. Serial selection and/or coinfection or other genomic modification(s) are preferably employed to drive the optimal conditions for bacterial proliferation in the direction of optimal host cell maintenance.

However once delivered to the target host, the host's immune systems are in play. The bacterial invader thus can effect killing both by its natural intracellular parasitism, but also if desired by selected or engineered features that mark the invaded cell's plasma membrane with a foreign bacterial signal protein. In these embodiments, both the intracellular proliferation and eventual lysis and the tagged plasma membranes serve as a multi-pronged attack on the cancer cell.

During selective development, bacteria can be co-cultured to speed exchange of adapted tropic receptors and cell killing methods. Preferably cell killing genes are engineered into the bacterial genome or at least carried as a stable plasmid. Classic molecular biology may be desired tropic strains may be applied to implement desired cell killing characteristics inside the selected tropic strains. Alternatively, genes responsible for the bacteria binding to the plasma membrane to effect entry into the cell may be engineered into killer bacteria.

The present invention features methods for selectively destroying abnormal cells, especially cancer cells or precancerous cells in a multi-cellular organism wherein the targeted cells are identified, e.g., marked by the adapted metabolisms inherent in growing cancer cells. Cells whose cancer adapted metabolism causes a local temperature increase and a local pH decrease allows binding of bacteria to these cells followed by bacteria integrating into the cytoplasm and proliferating within these cells. The bacteria may themselves consume and kill the cells or may initiate intracellular and/or extracellular immune responses.

As bacteria proliferate within a cell they normally lead to cell lysis and release of a population of additional bacteria that are capable of selectively destroying additional abnormal cells. The attacked cells alternatively may self-destruct though an intracellular immunity process that cells have developed to prevent infections from spreading to other cells. For example, apoptotic events can be used defensively by the organism to prevent growth of invading bodies and thereby halt the spread of infection. These events may involve mitochondrial activities including, but not limited to release of cytochrome c, reactive oxygen species and other anti-biologic or anti-cell constituents.

Infection may include activating a systemic immune response preferably invoking cross reactivity beyond the tagging instigated by biomolecules from the invading biologic. The systemic response may develop a humoral, for example antibody response and/or a cellular immunity response.

Bacteria may be produced in culture inside cells which then are lysed to release progeny bacteria, but preferably are produced in bacterial culture without need for a host eukaryotic cell. Such bacteria are commonly known and include, but are not limited to: bartonella henselae, brucella, francisella tularensis, legionella, listeria mono-cytogenes, salmonella typhi, mycobacterium, nocardia, rhodococcus equi and Yersinia. However, where eukaryotic culture may be desired, e.g., for regulatory simplicity, compliance, QC or other issues, non-facultative, e.g., bacteria growing within eukaryotic cell may be used. Eukaryotic culture may be accomplished in facultative cells, but may also be done using bacteria including, but not limited to: chlamydia, coxiella, rickettsia, etc.

In some embodiments a marker protein that cancer cells require for disposing of product of their lactate metabolism provides additional binding capacity. The monocarboxylate transporter 4 protein (MCT4) is generously present on surfaces of cancer cells because they need to remove lactic acid from cells. The MCT4 is the transport protein responsible for removing the lactate and its co-transported carbon which is responsible for the local pH decrease surrounding these cells.

Serial selection or other types of engineered virus or bacteria may serve as a source of proteins or the information for making or engineering proteins that can be incorporated in a liposomal membrane. While it is possible to favor orienting transmembrane protein particles so that a chosen portion predominates on the outer surfaces, simplified production with pseudorandom orientation will generally suffice given sufficient amounts of protein available for protein incorporation. Sufficiency requires only a small number of proteins to be exposed on the outer surface to bind the target moiety.
Fueling and Balancing Cells' Metabolisms

As living entities, cells require uncountable chemical reactions (metabolism) to synthesize and maintain their parts. To continue living they require raw materials to maintain function, to grow and to reproduce. Lone cells can obtain their nutrition from the immediate surroundings. But in complex organisms, where the cell may be distant from the outside environment a delivery service is necessary. In larger animals the circulatory system is responsible for delivering and clearing food and waste. A blood supply transgressing through a system of tubes (blood vessels) is used. As the organism grows each part must be supplied with appropriate blood vessels for support. The formation of blood vessels requires migration and proliferation of endothelial cells. These endothelial cells must be fueled in order to form and maintain the circulatory system.

The circulatory system is also an information system. Blood can carry chemical messages to and from the cells it services. The message does not need a locational address. Since cells are in contact with the environment (interstitial space) they are constantly removing chemicals from the space and depositing chemicals into it. The tools on the cell surface that help transport chemicals across the cytoplasmic membrane are exposed to the interstitial space. If a molecule has characteristic affinity for one of these “receptors” it will associate as a ligand with the receptor. A receptor may have one of many functional characteristics. It may serve to allow viral attachment to the cell membrane. It may act enzymatically to change the ligand in a manner including, but not limited to: isomerization, cleavage, covalent attachment, internalization (carry across the membrane), initiate encapsulation, present the ligand in receptive form to another ligand or receptor, etc. The receptor often will induce further changes inside the cell to manage (or metabolize) in some way the molecule being brought into the cell. While often signals are molecules manufactured by one cell and delivered to another to instruct that cell what it should do, simply classical food molecules can serve as signals to upregulate the pathways needed to metabolize that type of molecule.

Most cells ingest the chemical mass and energy they need to grow and proliferate in a form of carbon they find easy to use, e.g., amino acids (proteins) and sugars (carbohydrates).

However, when the cell is behaving in a specialized manner, the cell often must alter its pathways to support the specialized needs. Or in the chicken-egg question, when the cell has activated surprising metabolic pathways, then the cell will by necessity be doing something distinct from undifferentiated “normal” or “parent” cells.

For example, a growth signaling receptor protein when activated will initiate a signal cascade through to the cell nucleus to build food receptors and carriers and to transport these receptors and carriers to the plasma membrane. A sugar or amino acid then contacts the receptor and is carried inside. The carrier/transporter will initiate or activate an appropriate pathway inside the cell to metabolize the cargo. Perhaps the cargo is aminated or otherwise modified to divert to a less common metabolic pathway or to serve as an intracellular signal.
Acetyl CoA

One popular branching point, i.e., a molecule that might be directed through several metabolic pathways is acetyl Co-A. Often acetyl co-A is produced from the degradation of carbohydrates and/or proteins. But, especially in circumstances where nucleic acid synthesis is required (e.g., rapidly proliferating cells or cells expanding mitochondrial mass) fatty acids may become a favored source of carbon.

Acetyl-CoA is a lipogenic precursor for many lipid molecules including, but not limited to: isoprenoid, cholesterol and fatty acids. Another common precursor, oxaloacetic acid, which may also be directly exported from the TCA cycle, supplies pools of non-essential amino acids.
Mitochondria

Mitochondria are organelles in eukaryotic cells that are classically known for production of ATP from electron transfer (oxidation/reduction) reactions. The size and shape of mitochondria can vary within a single cell and each mitochondrial package may contain plural copies of the mitochondrial genome which is a double stranded circular DNA molecule that, for example in humans, encodes 37 genes. Mitochondria are dynamic organelles that can migrate within a cell along cytoskeleton framework. Mitochondria can grow by fusing with other mitochondria and may dissociate in a process termed fission that allows split up smaller mitochondrial bodies to move more freely and to different locations within the cell. The smaller mitochondria produced through fission have reduced distance for diffusion of substances they make and use. Mitochondria can grow by adding additional membrane and protein materials and may be digested through a process termed mitophagy or autophagy. In general, smaller mitochondrial bodies will have better communication with the cytoplasm due to reduced volume to surface ratio.

One target of cancer treatment could theoretically involve hindering the ability of cancer cell mitochondria to participate in either of these fusion or fission processes and thereby impact general mitochondrial functioning. However, accelerating the fission process in comparison to fusion may be one means through which neoplastic cells can diminish their death through apoptosis. Maintaining joined mitochondria as favored by fusion processes appears to make an apoptotic event more possible. Several proposed rounds for use in practicing the present invention emphasize maintenance of fused mitochondria. Mitochondria in cells are consistently changing. They are transported along the cytoskeleton to areas of need. They may change from more rodlike to more spherical shapes depending on location within a cell. During these processes, mitochondria may fuse together and may split apart under control of proteins within the cell. Several bacteria and viruses appear to apply such tactics during infection.

Two mitochondrial membrane proteins essential for mitochondrial fusion are mitofusin 1 (Mini) and mitofusin 2 (Mfn2) which connect two mitochondrial membranes as the fusion process begins. On the other side, another essential protein for maintaining healthy mitochondria is Drp1, a primarily cytosolic protein. When bound to a mitochondrion, Drp1 forms a constrictive ring around a mitochondrion to split it into two parts. Drp1 is one of the GTPase proteins in mammalian cells. Drp1 interacts with several proteins including, but not limited to: Fis1, Mff, MiD49 and MiD51, that act on the mitochondrial surface to initiate and control mitochondrial fission.

Fission is important for maintaining a healthy mitochondrial population and appears to be necessary for cells to proliferate. Fission often precedes mitosis, perhaps to make equal division easier. Drp1 activated mitochondrial fission is associated with inhibiting apoptosis, a property opposite that of eliminating the individual cell. Thus interfering with activity of any of these proteins may slow fission and maintain mitochondria in a fused state. Cancer cells are characterized by relatively fewer fused mitochondria with respect to more independent or smaller separate mitochondria than seen in non-malignant cells. Consistent with this observation is a finding that Drp1 expression is elevated in cancer cells and that the fraction of Drp1 phosphorylated at the serine residue at position 616 in Drp1, activated Drp1 is elevated. Apparently, cancer cells increase phosphorylation at this spot with the effect of favoring fission activities.

It is possible to chemically inhibit fission by interfering with Drp1. Mitochondrial division inhibitor 1 (Mdivi 1) is a quinazolinone derivative that selectively inhibits mitochondrial division by blocking dynamin GTPase activity in mammalian cells (IC₅₀=−50 μM). It has been shown to prevent apoptosis by inhibiting mitochondrial outer membrane permeabilization in vivo and to block Bid-activated Bax/Bak-dependent cytochrome c release from mitochondria in vitro.

Cayman Chemical reports that Mdivi 1 has been used to maintain mitochondrial integrity and to prevent cell death in models of pathological conditions including cancer, heart failure, and ischemia and reperfusion injuries. Another inhibitor of Drp1 is a compound known as P110. The polypeptide P110, DLLPRGT, appears more selective for blocking Drp1/Fist interaction than Drp1 interaction with other ligands. [A novel Drp1 inhibitor diminishes aberrant mitochondrial fission and neurotoxicity. Xin Qi, Nir Qvit, Yu-Chin Su, Daria Mochly-Rosen. J Cell Sci 2013 126: 789-802; doi: 10.1242/jcs.1144391. Delivering one or more Drp1 inhibitors in a chemical cocktail or biologically to the cancer cell targets can potentiate other pro-apoptotic interventions.

Bacteria are similar in structure to mitochondria and share many characteristics. These similarities, though not essential for intracellular parasitism, apparently contribute to successful parasitic maintenance in several cells.

Countering Cancer's Metabolic Changes

Cancer cells are distinguished from other cells usually based on their loss of controlled functions normally carried out by that organ or cell type and by their hyperproliferation. While the hyperproliferation can be understood from the viewpoint of the cell whose fittest life mission is to grow and continue its cell lineage, from the organism's point of view this group of rogue cells is not supportive of the survival life of the large organism: First, these cells are not performing activities for the good of the whole organism. Second, these cells are wasting nutrients. Third, the increased volume occupied by these cells interferes with communication and other functions of the non-cancer cells. Fourth, these cells are consuming (wasting) resources that could be more advantageously used. And fifth, these cells may be exporting toxic or problematic metabolites requiring surrounding tissues to expend resources and effort in clean-up operation.

Since the cells are performing different, i.e., abnormal, activities one would have to expect that reactions within cancer cells will be different from those within normal cells. To put it simply, different outputs and behaviors will require different activities to achieve them. The hyperproliferative action of the mutating or mutated cells will require an abundance of nutrients. The increased rate of reactions will produce excess metabolites, possibly abnormal metabolites, and will result in excess heat from the exothermic reactions which predominate in the general nature of reactions.

The cells will also differ in the way they utilize intracellular and extracellular nutrients. Addressing these differences provides strategies for impeding tumor growth and tumor cell proliferation. For example, as the cells hyperproliferate, pathways for manufacturing purines and pyrimidines for nucleic acids must be accelerated. Parasitic organisms will also require purine and pyrimidines either newly synthesized or ingested from intracellular stores or structures.

Glucose is a common fuel made by plants during photosynthesis and involved in multiple metabolic synthetic and fueling pathways. Enhanced glucose uptake required for hypermetabolism and hyperproliferation is a hallmark of several cancers and has been exploited in the clinic as a diagnostic tool through PET imaging of the glucose analogue 18F-deoxyglucose (18FDGPET). Moreover, in contrast to most normal tissues where much of the glucose is oxidized through the TCA cycle, in mitochondria, cancer cells preferentially convert glucose to lactate a three carbon molecule that retains and eventually removes energy unavailable for ATP synthesis. The fate of glucose inside cells is influenced by the enzymatic properties of the specific glycolytic gene products expressed. Expression of the M2 isoform of pyruvate kinase (PKM2) can contribute to the characteristic glucose metabolism of tumors and replacement of PKM2 with its splice variant PKM1 cannot efficiently support biosynthesis and tumor growth. Pyruvate kinase appears to be an important gateway in glucose metabolism that can be critical for controlling cell proliferation.

The aversion of cancer cells to the mitochondrial ETC and the conventional oxidative phosphorylation pathway for ATP production should be considered a requirement, not an anomaly of cancer cells. Remember that these cells were once considered “normal” cells but in their progression to the hyperproliferative state have had to alter normal cell functions. The hyperproliferation would be expected to change many metabolic pathways to support the new activities. These abnormal pathways would be expected to require abnormal raw materials or amounts of raw materials in the nutrients consumed or in the metabolic intermediates necessary to sustain the new way of life for the cell. It is thus wise to think of the altered metabolism, not as a symptom of cancer, but as one of the links in the causative chain.

Most cancers are believed to initially present with a genetic abnormality, many inherited or virally introduced. Several genes have known alleles that support or initiate development of cancer. For example, greater than 50 cancers are associated with genes that increase cancer risk in individuals inheriting one or more copies from a parent. BRCA1 and BRCA2 (associated with breast cancer), TP53 (Li-Fraumeni syndrome), and PTEN (Cowden Syndrome) are some well-publicized cancer risk genes. Several viruses, e.g., human cytomegalovirus, Epstein-Barr virus, human papillomavirus, hepatitis B virus, and hepatitis C virus are also genetic factors that increase cancer risk, i.e., events contributing to supporting a cancer cell's metabolic transformations.

An external event switching a gene on or off may initiate or contribute to the cancer cascade. If the organism is inattentive to the changing cell, the cell may be allowed to continue development to a cancerous status. But to support the change the cell will have to adapt. Humans and other mammals have evolved means to halt the requisite adaptations. For example, either inherited or somatic mutations of TP53, a tumor suppressor gene for p53 protein, removes a brake on growth of abnormal cells and allows the metabolic transformations necessary for cancer cell proliferation to proceed. Another gene with tumor suppressive activity and whose mutation removes restraints on uncontrolled growth is CHEK2.

Some adaptations will be built in, in accordance with feedback loops that evolution has given us; some may involve additional mutations in the nuclear or mitochondrial genomes; some may be more complex evolved responses, for example, an epigenetic modification like methylation.

At the base of cell growth is metabolism and nutrition supporting the metabolism. The variety of underlying causes and adaptations supporting the initial events may require a variety of routes to counter the metabolic signature of a cancer cell. But all routes will to some extent address the abnormal metabolisms.

One simple course of treatment will be to support “normal” metabolism. That is to provide raw material (nutrients) supporting normal metabolism, for example to favor ETC activity. In concert with this can be a restriction on types of raw materials supporting the diverted or cancer enhanced or enhancing metabolic pathways. A more aggressive strategy may include inhibitors of one or more of these side pathways. When these cells are deprived of the environment in which they mutated and may have in fact contributed to, selective pressure will tilt against these cells in favor of the “normal” cells.

Nutrition can also be altered with a goal of supporting apoptotic activity and inhibiting cells that counter apoptosis. These and other healthy supports may be used in series or in parallel with bacterial strategies similar to those set forth in this description

Genetic Intervention

Besides simply altering nutrition needs, in many cases leading to cancer the gene expression will have been irreversibly altered. These modifications, whether in primary sequence or epigenetic modifications offer a grand opportunity for treatment. Genetic engineering tools can recognize specific mutations and when coordinated with an endonuclease can remove or edit identified genetic abnormalities. Systems such as CRISPR have recognized ability to distinguish methylated from non-methylated bases in genetic sequence.

Gene editing processes are continually being improved. To date they have improved precision and specificity and become acceptable in practice. An example of a recent summary of technology appears in US patent Application 20170035860.
Gene editing technologies: Recent developments of technologies to permanently alter the human genome and to introduce site-specific genome modifications in disease relevant genes lay the foundation for therapeutic applications in CNS disorders such as Parkinson's disease (PD) or Alzheimer disease (AD). These technologies are now commonly known as “genome editing.” Current gene editing technologies comprise zinc-finger nucleases (ZFN), TAL effector nucleases (TALEN), and clustered regularly interspaced short palindromic repeats (CRISPR)/CRISPR-associated (Cas) system ora combination of nucleases (e.g. mutated Cas9 with Fok1) (Tsai, S. Q., Wyvekens, N., Khayter, C., Foden, J. A., Thapar, V., Reyon, D., Goodwin, M. J., Aryee, M. J., and Joung, J. K. (2014). Dimeric CRISPR RNA-guided Fold nucleases for highly specific genome editing. Nature biotechnology 32, 569-576.)) All three technologies create site-specific double-strand breaks. The imprecise repair of a double strand break by non-homologous end joining (NHEJ) has been used to attempt targeted gene alteration (nucleotide insertion, nucleotide deletion, and/or nucleotide substitution mutation). A double-strand break increases the frequency of homologous recombination (HR) at the targeted locus by 1,000 fold, an event that introduces homologous sequence at a target site, such as from a donor DNA fragment. Another approach to minimize off-target effects is to only introduce single strand breaks or nicks using Cas9 nickase (Chen et al., 2014; Fauser et al., 2014; Rong et al., 2014; Shen et al., 2014).
The CRISPR/Cas9 nuclease system can be targeted to specific genomic sites by complexing with a synthetic guide RNA (sgRNA) that hybridizes a 20-nucleotide DNA sequence (protospacer) immediately preceding an NGG motif (PAM, or protospaceradjacent motif) recognized by Cas9. CRISPR-Cas9 nuclease generates double-strand breaks at defined genomic locations that are usually repaired by non-homologous end-joining (NHEJ). This process is error-prone and results in frameshift mutation that leads to knock-out alleles of genes and dysfunctional proteins (Gilbert et al., 2013; Heintze et al., 2013; Jinek et al., 2012). Studies on off-target effects of CRISPR show high specificity of editing by next-generation sequencing approaches (Smith et al., 2014; Veres et al., 2014) (FIG. 1, panel 1).
Other applications for heart disease, HIV, and Rett syndrome have been described. (Ding et al., 2014; Swiech et al., 2014; Tebas et al., 2014). For heart disease, permanent alteration of a gene called PCSK9 using CRIPR technology reduces blood cholesterol levels in mice (Ding et al., 2014). This approach was based on the observation that individuals with naturally occurring loss-of-function PCSK9 mutations experience reduced blood low-density lipoprotein cholesterol (LDL-C) levels and protection against cardiovascular disease (Ding et al., 2014). A second example for the feasibility of this approach is HIV. Individuals carrying the inherited Delta 32 mutation in the C-C chemokine receptor type 5, also known as CCRS or CD195 are resistant to HIV-1 infection. Gene modification in CD4 T cells were tested in a safety trial of 12 patients and has shown a significant down-regulation of CCR5 in human (Tebas et al., 2014). Another recent study showed the successful use of CRISPR/Cas9 technology in CNS in a mouse model for the editing of the methyl-binding protein 2 (MecP2) gene. Mutation in this gene causes Rett syndrome, a condition in young children—mostly girls—with mental retardation and failure to thrive. In this approach an adenoassociated virus (AAV) was used as the delivery vehicle for the Cas9 enzyme in vivo. Overall, 75% transfection efficiency was described with a high targeting efficiency that almost completely abolished the expression of MecP2 protein and functionally altered that arborization of the neurons similar to what has been described for Rett syndrome (Swiech et al., 2014). This shows the proof of concept that gene editing using CRISPR/Cas9 technology is achievable in the adult brain in vivo.
Despite reports in the literature describing the use of genetic editing techniques, none have been described or suggested for genes associated with neurodegenerative disorders. A strong need continues to exist in the medical arts for a method for treating and/or inhibiting diseases associated with neurodegenerative disorders, such as materials and techniques useful for the treatment of Parkinson's Disease.

### SUMMARY OF THE INVENTION

In a general and overall sense, the present invention provides for the arrest and/or prevention of neurodegeneration associated with neurodegenerative disease in vivo. In some embodiments, arrest and/or prevention of neurodegeneration is accomplished using gene editing methodologies and molecular tools to manipulate specific gene(s) and/or gene regulatory elements, to provide a modification of the gene and/or genomic regions associated with neurodegeneration and neurodegenerative disease, such as Parkinson's Disease.
In some aspects, the present invention provides a method of treating a neurological deficit associated with neuropathological disease comprising administering a genetically engineered vector comprising a gene for a nuclease and a promoter for the nuclease, as well as an appropriate molecular “guide” into a cell. Following the administration, the vector facilitates an expression of a molecular component that alters a gene in the cell or expression of a targeted gene associated with the neuropathology in the cell. The affected gene would be implicated in an etiology of the neurological deficit.
In other embodiments, a medical composition for treating a neurological deficit in a patient is provided. The medical composition includes a nuclease that introduces double strand break in a gene implicated a neurological deficit, a guide RNA that targets a gene implicated in neurological disease, and a delivery system that delivers the nuclease and guide RNA to a cell.
For purposes of the description of the present invention, the term “modification of gene and/or genomic region” may be interpreted to include one or more of the following events (FIG. 1):
a) Targeted introduction of a double-strand break by a composition disclosed, resulting in targeted alterations (random mutations e.g. insertions, deletions and/or substitution mutations) in one or more exons of one or more genes. This modification in some embodiments provides a permanent mutation in a cell or population of cells having the modified gene.
b) Targeted binding of non-functional mutant Cas9 to non-coding regions (e.g. promoters, evolutionary conserved functional regions, enhancer or repressor elements). Binding is induced by compositions disclosed. Sterical hindrance of binding of other proteins (e.g. transcription factors, polymerases or other proteins involved in transcription) may also result as a consequence of binding.
1. CRISPR sgRNA introduces small insertions or deletions through non-homologous end joining (NHEJ), in general several nucleotides, rarely larger fragments (Swiech et al., 2014).
2. Homology-directed repair (HDR) to correct point mutations by introducing a non-natural, but partially homologous template.
3. Double Genome editing of splice-sites or splicing related non-coding elements to eliminate certain gene regions, e.g. exon 5 of SNCA gene.
4. Double Genome editing of non-coding or intronic gene regions to eliminate regulatory elements that increase or decrease gene expression, e.g. D6 or 112 regulatory region in SNCA gene.
5. sgRNA guides mutant Cas9 to physically inhibit binding of transcription factors in promoter region.
6. sgRNA guides mutant Cas9 to physically inhibit binding of transcription factors in regulatory regions or intronically.
Gene editing or modification can be achieved by use of any variety of techniques, including zinc-finger nuclease (ZFN) or TAL effector nuclease (TALEN) technologies or by use of clustered, regularly interspaced, short palindromic repeat (CRIPSR)/Cas9 technologies or through the use of a catalytically inactive programmable RNA-dependent DNA binding protein (dCas9) fused to VP16 tetramer activation domain, or a Krueppel-associated box (KRAB) repressor domain, or any variety of related nucleases employed for gene editing. These can be seen as existing tools to sever the genomic region in question.
The tools mentioned above, are general in their application. Aspects of the present methods and compositions provide the design of custom CRISPR single-guide RNA (sgRNA) sequences specific for coding gene regions and regulatory sequences in genes implicated in neurodegeneration. In this manner, an exact genomic location for precise gene alteration in humans may be accomplished, with a resulting improvement and/or elimination of a neurodegenerative disorder pathology or symptom.

Additional patents and patent applications, for example, US application number 20170015994 evidence the utility, feasibility and enablement of gene editing processes with high specificities are well known and accepted in the art. Thus, complex tools for recognizing aberrant metabolisms are recognized as potential targets for treating cancers more or less specific to recognized genetic underpinnings. Specific genes may be addressed accordingly. However, in accordance with the present invention, though possible for use in conjunction with such targeted strategies, a broader, more universally effective, therapy is available.

Those these identified genetic abnormalities can thus be considered targets that are recognizable by some very specific tools, in general these targets must be confirmed for individual cancers. Depending on the identified modified gene, different strategies are proposed as available for treating these specific cells. The DNA might simply be cut and irreversibly capped to prevent further mitosis by that cell. Incorrect genes might be turned on, for example, to initiate cell division before the genome had been copied, to activate genes incompatible with continued viability of the cell, to correct the gene abnormality, to permanently turn off the gene. These and similar strategies would increase stress on the cells, especially cells expressing the targeted genetic modification, and even if not fatal to the cell will reduce its fitness and survivability. Viruses, especially retro-viruses can have great affect on temporary and permanent genomic activities. While bacteria do not integrate their genomes into the hosts genome, their metabolisms and secreted enzymes are powerful forces during parasitic growth and eventual lysis.

When a cell characteristic can be targeted, e.g., a Ras expressing cell targeted by a virus, the weapon might be factors to turn on, activate, augment, or duplicate activity of desired proteins. These can be proteins supporting and restoring more normal metabolism, but might also be proteins supporting cell death, for example proteins supporting initiation or progression of apoptosis. On the flip side, anti-apoptotic protein activity or expression might be blocked.

Transcription factors or other manipulation of transcription may be used to increase expression of a protein or to throttle it down. The targeted gene need not be a gene mutated in the cancer process, so long as the cellular process is acceptably targetable. These might be protein or nucleic acid based and could be directed against a modified gene, of course, or could be targeted against a more ubiquitously required or associated gene necessary to accomplish or prevent a proliferation event. Genes involved in the cell cycle, genes involved in cytoskeleton, genes required for membrane integrity, genes required for any cellular or subcellular process, etc., essentially any well used or essentially expressed gene might be selected for the ultimate target. RNAi can be used to inhibit transcription and therefore protein activity. DNA or modified DNAs may be incorporated into genomic material of the nucleus or mitochondrion. RNAi molecules can be put to assorted applications including interruption of protein translation.

Species of RNAi, miRNA is encoded in nuclear DNA and several viruses as a means for turning off translation of messenger RNA (mRNA) molecules. Though shorter than common synthetic siRNA constructs their actions are similar to those of siRNA in ability to turn off protein production. But because of their shorter region of complementarity miRNAs often impact expression of several, possibly hundreds of mRNAs. Nuclear DNA includes portions that encode for miRNA. These portions may be found in either introns or exons and in many cases are constitutively expressed. Once in the cytoplasm miRNAs appear to have half-lives many fold longer (some estimate −10-fold longer) than their targeted mRNAs. The applicability exogenous miRNA for controlling cell function is evidenced by the ability of many viruses to use miRNAs to shut off a cell's anti-viral defenses. Engineering a viral vector to include desired miRNA precursors can be useful for controlling expression of most proteins, including especially mitochondrial proteins.

Short interfering RNA (siRNA) are double stranded self-complementary RNA molecules popular for use in research and genetic therapeutic applications. The siRNA is stabilized by proteins in a RSC complex within the cell to present a single stranded region available for complementary binding with mRNA. Once bound the mRNA is cleaved thereby preventing its translation to polypeptide and marking it for degradation. Therapists have preferred siRNA over miRNA because of siRNA's shorter half-life and because its targets are more limited and therefore specific.

Synthetic siRNAs are deliverable to cells by methods known in the art, including, but not limited to: including cationic liposome transfection and electroporation. Generally, exogenous siRNA have short term persistence of the silencing effect limiting risk of long term off-target effects. For longer term effect siRNA or miRNA can be incorporated into a host genome through genetic engineering.

Genetic engineering can include miRNAs that down-regulate gene expression at the post transcriptional or translational level. Engineering may include substituting the native sequences of the miRNA precursor with miRNA sequence complementary to a target mRNA encoding any carrier, enzyme, receptor, pore, etc. of choice. The vectors delivering the novel or foreign miRNA can be used to produce siRNAs to initiate interference against specific mRNA targets.

Genetic engineering is a rapidly developing art increasingly including post transcription mechanisms of action. Examples include chemically modified siRNAs or short interfering nucleic adds (siNAs) as revealed in US Patents and Patent Applications such as: 20160244760, 20160053269 RNA Interference Mediated Inhibition Of Gene Expression Using Chemically Modified Short Interfering Nucleic Add (siNA), 20170022146 Novel Low Molecular Weight Cationic Lipids For Oligonucleotide Delivery (SIRNA Therapeutics (Merck), now owned by Anylam); 20160331828, 20160317647 Nucleic add Vaccines, 9464124, 20160271272 Engineered Nucleic Adds And Methods Of Use Thereof, 20160244501 Polynucleotides Encoding Low Density Lipoprotein Receptor; U.S. Pat. Nos. 9,295,689, 9,271,996 Formulation and delivery of PLGA microspheres, 9,254,311 Modified polynucleotides for the production of proteins, 9,283,287 Modified polynucleotides for the production of nuclear proteins (ModeRNA Therapeutics); 20170044239 Phage-Displayed Antibody Libraries And Uses Thereof (Academia Sinica); 20170037431 In vivo Gene Engineering with Adenoviral Vectors (University of Washington); 20170044541 miRNAs Enhancing Cell Productivity (1-lochschule Biberach); 20170044555 Recombinant RNA Particles And Methods Of Producing Proteins (Synthetic Genomics, Inc.). Viruses may be specifically engineered for identified cancers and may benefit from improved targeting at cells expressing higher temperature or excreting exaggerated amounts of hydrogen ion. Bacteria, with their own RNAses and binding proteins also impact the host cell genome and ability to continue growth.

Pyruvate and Lactate

Pyruvate kinase catalyzes the last step of glycolysis, transferring the phosphate from phosphoenolpyruvate (PEP) to adenosine diphosphate (ADP) to yield adenosine triphosphate (ATP) and pyruvate. In mammals, two genes encode a total of four pyruvate kinase isoforms. The Pkrl gene encodes the PKL and PKR isoforms, expressed in the liver and red blood cells respectively. Either the PKM1 or PKM2 isoform encoded by the Pkm gene is found in cells. PKM1 is found in many normal differentiated tissues whereas the PKM2 is expressed in most proliferating cells including all cancer cell lines and tumors tested. PKM1 and PKM2 are derived from alternative splicing of a Pkm gene transcript by mutual exclusion of a single conserved exon that encodes 56 amino acids. Despite the similar primary sequences, PKM1 and PKM2 have different catalytic and regulatory properties. PKM1 appears always active, exhibiting high constitutive enzymatic activity. In contrast, PKM2 is less active, but is allosterically activated by the upstream glycolytic metabolite, fructose-1,6-bisphosphate (FBP).Unlike other pyruvate kinase isoforms, PKM2 can interact with proteins harboring phosphorylated tyrosine residues thereby releasing FBP which, in a feedback mechanism, reduces the activity of the enzyme. Low PKM2 activity, in conjunction with increased glucose uptake, facilitates use of glucose carbons into anabolic pathways derived from glycolysis. Also, PKM2, but not PKM1, can be inhibited by direct oxidation of its cysteine 358 as an adaptive response to increased intracellular reactive oxygen species (ROS).

Additionally, PKM2 expression in cancer cells has been associated with enhanced phosphorylation of the H₁₁ on phosphoglycerate mutase 1 (PGAM1) by PEP. This pathway is an alternative route for pyruvate production but bypasses the generation of ATP via the pyruvate kinase step. This supports high rates of glycolysis. Replacement of PKM2 with the constitutively active isoform PKM1 results in reduced lactate production, enhanced oxygen consumption, and a decrease in PGAM1 phosphorylation. There also appears to be selection for PKM2 expression for growth in vivo. Alternatively, PKM2 expression may evidence selection against high pyruvate kinase activity and therefore against expression of PKM1. This rationale suggests that activation of PKM2 may impede cancer cell proliferation by interfering with regulatory mechanisms critical for proliferative metabolism.

It is expected that PKM2 activators will mimic the regulatory properties of constitutively active PKM1, thereby promoting high PKM2 activity regardless of the known mechanisms cells use to decrease pyruvate kinase activity. Similar to results observed when PKM2 is replaced with PKM18, under standard tissue culture conditions, PKM2 activators had no significant effects on cell proliferation when tested across several lines. In contrast, when proliferation is assessed under hypoxic conditions (˜1% 0₂), PKM2 activator treatment results in decreased rate of cell proliferation in comparison to DMSO-treated cells. And expression of PKM1 in the presence of endogenous PKM2 has no effect on cell proliferation in standard tissue culture conditions, but inhibits proliferation under hypoxia to a similar degree as treatment with PKM2 activators. Replacement of PKM2 with PKM1 also impairs cell proliferation under hypoxic conditions.

Cancer cells harbor genetic changes that allow them to increase nutrient uptake and alter metabolism to support anabolic processes, and interfering with this metabolic program is a viable strategy for cancer therapy. Altered glucose metabolism in cancer cells is mediated in part by expression of PKM2, which has specialized regulatory properties. Unlike its splice variant PKM1, which is found in many normal tissues, PKM2 is allosterically activated by FBP and can interact with tyrosine-phosphorylated proteins to release FBP and decrease enzyme activity.

Thus, growth factor signaling promotes decreased PKM2 activity and availability of glycolytic metabolites for anabolic pathways that branch from glycolysis. This suggests that activation of PKM2 might oppose the effects of growth signaling and interfere with anabolic glucose metabolism.

In this situation where pyruvate kinase activation has occurred, high pyruvate kinase activity, for example, associated with a bacterial infection, would suppress tumor growth.
Mitochondrial Genome

Mitochondria have a limited set of genes in their genome. Most proteins in the mitochondrial membranes and matrix are encoded in the nuclear genome before being translated on the cytoplasmic ribosomes. These nuclear encoded mitochondrial genes include, but are not limited to: mitochondrial enzymes, mitochondrial membrane pore and carrier proteins and chaperone or folding proteins.

The mitochondrial genome consists of one double stranded DNA polymer in a circular format, i.e., no apparent beginning or end. Mitochondrial genes can code for RNA or polypeptide polymers. The 37 mitochondrial genes are split between the two complementary DNA strands. A strand with higher guanine cytosine ration is called the H-strand and the complement is dubbed the L-strand. The H-strand is richer in genes with twenty-eight of the thirty-seven. L-strand genes include TRNA, TRNC, TRNE, TRNY, TRNN, TRNP, TRNS1, ND6 AND CR; while the H-strand genes encode TRNT, CYTB, NDS, TRNL2, TRN2, TRNH, ND4, ND4L, TRNR, ND3, TRNG, COX3, ATP6, ATPS, TRNK, COX2, TRND, COX1, TRNW, ND2, TRNM, TRNI, ND1, TRNL1, RRNL, TRNV, RRNS and TRNF. A typical cell will contain between 10² and 10⁴ DNA molecules (paired strands). However, sex cells vary with the egg carrying “2×10⁵ and sperm bringing 10¹ or fewer. Typically perhaps 15 mitochondria may harbor up to 500 genome molecules total. But numbers vary with cell type and with time in a given cell.

Most mitochondrial genes encode molecules that remain in the mitochondrion. Only humanin (an anti-apoptotic protein) is mtDNA encoded (by the larger ribosomal RNA encoding gene), but humanin is exported from the mitochondrion and exerts its effects after release into the cytoplasm. The mitochondrion has its own ribosomal RNAs (2) and tRNAs (22). Leucine and serine each have two tRNAs.

Mitochondrial proteins encoded by mitochondrial DNA (mtDNA) are involved in the ETC which has five complexes: NADH:ubiquitone reductase, succinate dehydrogenase, cytochrome k_{J}., cytochrome c oxidase and ATP synthase. Each of these complexes resides in the inner mitochondrial membrane.

Seven Complex 1 protein subunits are encoded in mtDNA: ND1, ND2, ND3, ND4, ND4L, ND5 and ND6. Thirty-eight additional protein complex subunits are encoded in the cell nucleus. Two copies of NDUFAB1 are in a complex 1 assembly, but every other subunit: NDUFA2, NDUFA6, NDUFA7, NDUFAI2, NDUFB1, NDUFB4, NDUFB6, NDUFV1, NDUFV2, NDUFV3 (in the N module); NDUFAS, NDUFS2, NDUFB3, NDUFB7, NDUFS8 (in the Q module); MT-ND1, NDUFA3, NDUFA6, NDUFA13, (in the ND module); MT-ND2, MTND3, NDUFA1, NDUFA10, NDUFC1, NDUFC2, NDUFS5 (in the ND2 module); MT-ND4, NDUFB1, NDUFBS, NDUFB10, NDUFB11 (in the ND4 module); MT-ND5, NDUFB3, NDUFB7, NDUFB8, NDUFB9 (in the ND5 module); NDUFA11 (possibly bridging the Q and ND1 modules); and NDUFAB1, NDUFA9, NDUFB4, NDUFB6 (uncertain module assignment) is only present as a single copy. Complex 1 assembly is facilitated or chaperoned by assembly factors including, but not limited to: Ndufaf3 (C3orf60), Ndufaf4 (C6orf66), Ndufaf1 (CIA30), C20orf7, Ecsit, Ind1 and Ndufaf2 (B17.2L).

Other proteins encoded by nuclear DNA but transported into the mitochondria include but are not limited to: phosphoenolpyruvate carboxykinase, hinge protein (fragment), 14-3-3 protein epsilon, tryptophanyl-tRNA synthetase, VDAC4 protein (Fragment), voltage-dependent anion-selective channel protein 3, voltage-dependent anion channel (fragment), voltage-dependent anion-selective channel protein 2 , voltage-dependent anion-selective channel protein 1, vesicle-associated membrane protein 1 (VAMP-1) (synaptobrevin 1), ubiquinol-cytochrome C reductase complex 11 kDa protein, ubiquinol-cytochrome C reductase iron-sulfur subunit, ubiquinol-cytochrome C reductase complex core protein 2, ubiquinol-cytochrome C reductase complex core protein I, ubiquinol-cytochrome C reductase complex 14 kDa protein, ubiquinol-cytochrome C reductase complex 7.2 kDa protein, Uracil-DNA-glycosylase, uracil-DNA glycosylase, mitochondrial precursor (UDG), mitochondrial uncoupling protein 4 (UCP4), mitochondrial uncoupling protein 3 (UCP3), mitochondrial uncoupling protein 2 (UCP2) (UCPH), mitochondrial brown fat uncoupling protein 1 (UCP1) (thermogenin), thioredoxin reductase 2, thioredoxin, mitochondrial translation elongation factor EF-Tu (fragment), elongation factor Tu, thiosulfate sulfurtransferase (rhodanese), elongation factor Ts, heat shock protein 75 kDa, DNA topoisomerase I, mitochondrial precursor proteins import receptor (translocase of outer membrane TOM70), probable mitochondrial import receptor subunit TOM40 homolog, mitochondrial import receptor subunit TOM20 homolog, probable mitochondrial import receptor subunit TOM7 homolog, mitochondrial import receptor Tom22, trimethyllysine dioxygenase, thymidine kinase 2, thymidine kinase, mitochondrial import inner membrane translocase subunit TIM9 A, mitochondrial import inner membrane translocase subunit TIM8 B, mitochondrial import inner membrane translocase subunit TIM8 A, Import inner membrane translocase subunit TIM44, mitochondrial import inner membrane translocase subunit TIM23, mitochondrial import inner membrane translocase subunit TIM22, mitochondrial import inner membrane translocase subunit TIM17 B (JM3), mitochondrial import inner membrane translocase subunit TIM17 A, mitochondrial import inner membrane translocase subunit TIM13 B, mitochondrial import inner membrane translocase subunit TIM13 A, mitochondrial import inner membrane translocase subunit TIM10, tumorous imaginal discs homolog precursor, transcription factor 1, putative ATP-dependent mitochondrial RNA helicase, surfeit locus protein 1, sulfite oxidase, succinyl-CoA ligase [GDP-forming] β-chain, succinyl-CoA ligase [GDP-forming] α-chain, succinyl-CoA ligase [ADP-forming] β-chain, steroidogenic acute regulatory protein, single-stranded DNA-binding protein, succinate semialdehyde dehydrogenase, superoxide dismutase [Mn], Smac protein, sodium/hydrogen exchanger 6 (Na+/H+ exchanger 6) (NHE-6), ADP/ATP carrier protein, liver isoform T2 (ADP/ATP translocase 3), ADP/ATP carrier protein, fibroblast isoform (ADP/ATP translocase 2), ADP/ATP carrier protein, heart/skeletal muscle isoform T1 (ADP/ATP translocase 1), Phosphate carrier protein, mitochondrial precursor (PTP), mitochondrial 2-oxodicarboxylate carrier, mitochondrial carnitine/acylcarnitine carrier protein, mitochondrial deoxynucleotide carrier, solute carrier family 25, member 18, peroxisomal membrane protein PMP34, mitochondrial ornithine transporter 1, brain mitochondrial carrier protein-1 (BMCP-1), calcium-binding mitochondrial carrier protein Aralar2, calcium-binding mitochondrial carrier protein Aralar1, mitochondrial 2-oxoglutarate/nnalate carrier protein (OGCP), mitochondrial dicarboxylate carrier, tricarboxylate transport protein, serine hydroxynnethyltransferase, serine hydroxynnethyltransferase, cytosolic, sideroflexin 3, sideroflexin 2, sideroflexin 1, oligoribonuclease, succinate dehydrogenase (ubiquinone) cytochrome B small subunit, succinate dehydrogenase cytochrome b560 subunit, succinate dehydrogenase (ubiquinone) iron-sulfur protein, succinate dehydrogenase (ubiquinone) flavoprotein subunit, nonspecific lipid-transfer protein, SCO2 protein homolog, mitochondrial precursor, SCO1 protein homolog, mitochondrial precursor, seryl-tRNA synthetase, Reticulon 4 (Neurite outgrowth inhibitor), 40S ribosomal protein S3a, mitochondrial 28S ribosomal protein S21 (MRP-S21) (MDS016), 28S ribosomal protein 517, mitochondrial precursor (MRP-S17)(HSPC011), 28S ribosomal protein 516, mitochondrial precursor (MRP-S16) (CGI-132), 28S ribosomal protein 515, mitochondrial precursor (MPR-S15) (DC37), 2-5A-dependent ribonuclease, NADH dehydrogenase subunit 3 homolog/ND3 homolog (fragment), delta 1-pyrroline-5-carboxylate synthetase (PSCS), Serine protease HTRA2, Lon protease homolog, proline oxidase, peroxiredoxin 5, thioredoxin-dependent peroxide reductase, protoporphyrinogen oxidase, peptidyl-prolyl cis-trans isomerase, Inorganic pyrophosphatase 2, DNA-directed RNA polymerase, DNA polymerase γ subunit 2, DNA polymerase γ subunit 1, ARTS protein, mitochondrial processing peptidase β subunit, mitochondrial processing peptidase α subunit, paraplegin (spastic paraplegia protein 7), probable glutamyl-tRNA(Gln) amidotransferase subunit B, phosphatidylethanolamine N-methyltransferase, pyruvate dehydrogenase protein X component, [pyruvate dehydrogenase [lipoamide]]-phosphatase 2, [pyruvate dehydrogenase [lipoamide]]-phosphatase 1, [pyruvate dehydrogenase [lipoamide]] kinase isozyme 4, [pyruvate dehydrogenase [lipoamide]] kinase isozyme 3, [pyruvate dehydrogenase [lipoamide]] kinase isozyme 2, [pyruvate dehydrogenase [lipoamide]] kinase isozyme 1, pyruvate dehydrogenase El component β subunit, Pyruvate dehydrogenase El component a subunit, testis-specific form, Pyruvate dehydrogenase El component a subunit, somatic form, pyruvate dehydrogenase El-α-subunit (fragment), pyruvate dehydrogenase El-α-subunit (fragment), pyruvate dehydrogenase El-α-subunit (fragment), pyruvate dehydrogenase El-α-subunit (fragment), pyruvate dehydrogenase El-α-subunit (fragment), pyruvate dehydrogenase El-α-subunit (fragment), peptide deformylase, mitochondrial 28S ribosomal protein S30 (MRP-S30), programmed cell death protein 8, phosphoenolpyruvate carboxykinase, mitochondrial precursor [GTP], phosphoenolpyruvate carboxykinase, cytosolic [GTP], propionyl-CoA carboxylase β chain, propionyl-CoA carboxylase a chain, pyruvate carboxylase, transmembrane protein, succinyl-CoA:3-ketoacid-coenzyme A transferase, cytochrome oxidase biogenesis protein OXA1, ornithine carbamoyltransferase, mitochondrial ornithine transporter 2, optic atrophy 3 protein, dynamin-like 120 kDa protein, mitochondrial outer membrane protein 25, N-glycosylase/DNA lyase, 2-oxoglutarate dehydrogenase El component, ornithine aminotransferase, nuclear respiratory factor-1 (NRF-1), NAD(P) transhydrogenase, nucleoside diphosphate kinase, neurolysin, mitochondrial precursor, NOGO-interacting mitochondrial protein, cysteine desulfurase, NADH-ubiquinone oxidoreductase 9 kDa subunit, NADH-ubiquinone oxidoreductase 24 kDa subunit, 24-kDa subunit of complex 1 (fragment), NADH-ubiquinone oxidoreductase 51 kDa subunit, NADH-ubiquinone oxidoreductase 23 kDa subunit, NADH-ubiquinone oxidoreductase 20 kDa subunit, NADH-ubiquinone oxidoreductase 13 kDa-A subunit, NADH-ubiquinone oxidoreductase 15 kDa subunit, NADH-ubiquinone oxidoreductase 18 kDa subunit, NADH-ubiquinone oxidoreductase 30 kDa subunit, NADH-ubiquinone oxidoreductase 49 kDa subunit, NADH-ubiquinone oxidoreductase 75 kDa subunit, NADH-ubiquinone oxidoreductase subunit B14.5b, NADH-ubiquinone oxidoreductase KFYI subunit, NADH-ubiquinone oxidoreductase B22 subunit, NADH-ubiquinone oxidoreductase ASHI subunit, NADH-ubiquinone oxidoreductase B18 subunit, NADH-ubiquinone oxidoreductase B17 subunit, NADH-ubiquinone oxidoreductase SGDH subunit, NADH-ubiquinone oxidoreductase B15 subunit, NADH-ubiquinone oxidoreductase B12 subunit, NADH-ubiquinone oxidoreductase AGGG subunit, NADH-ubiquinone oxidoreductase PDSW subunit, NADH-ubiquinone oxidoreductase MNLL subunit, Acyl carrier protein, NADH-ubiquinone oxidoreductase 39 kDa subunit, NADH-ubiquinone oxidoreductase 19 kDa subunit, NADH-ubiquinone oxidoreductase subunit B14.5a, NADH-ubiquinone oxidoreductase B14 subunit, NADH-ubiquinone oxidoreductase 13 kDa-B subunit, NADH-ubiquinone oxidoreductase MLRQ subunit, NADH-ubiquinone oxidoreductase B9 subunit, NADH-ubiquinone oxidoreductase B8 subunit, NADH-ubiquinone oxidoreductase 42 kDa subunit, NADH-ubiquinone oxidoreductase MWFE subunit, and NADH dehydrogenase subunit 6. These proteins have had their genes sequenced and are known in the art.

Modifying a cell's nuclear DNA is actually more straight forward than modifying a cell's mitochondrial DNA simply because of the number of relevant genes in a cell. Nuclear genes have but two alleles, one allele on each half of the pared chromosomes. Mitochondrial genes in a single cell are much more abundant; an individual mitochondrion may have several dozen circular genomes; and each cell can have a dozen or more mitochondria. Because mitochondria are continuously fusing with other mitochondria each mitochondrion may include plasmic copies. Many cancer cells present with homoplasmic mtDNA mutations. This suggests that the mutated mtDNA rendered significant survival benefits to the homoplasmicly mutated cell. Given the environment in the e.g., hyperproliferating cancer cell, the homoplasmic mitochondrial mutations when paired with the cell's other metabolic deviations were strong supporters of survivability of those mitochondria and of the cell hosting them.

Since the nucleus and mitochondria contain DNA as their genetic material similar genetic engineering principles can apply. Gene editing involves excising, inserting or substituting one or more genes or epigenetic modification of a gene, i.e., modifying a gene sequence or modifying ability of a transcription factor to bind and initiate or halt a gene's transcription.

Excising a gene will require the DNA molecule to be cleaved at the beginning and end of the DNA strand being removed. Insertion requires but one cleavage point with each end of the opening being compatible (usually short complementary overlapping single stranded endpoints). Substitution events require both excision and insertion. Sometime the excision and insertion sites are identical, but this is not an absolute requirement.

DNA molecules are nucleotide acids and are cleaved by nuclease enzymes (nucleases). Four classes of nuclease have been employed extensively in genetic engineering: meganucleases, zinc finger nucleases (ZFNs), transcription activator-like effector-based nucleases (TALEN), and the CRISPR-Cas system.

Gene editing systems can be made specific to mutated sequences, including epigenetic mutations. In cells then only undesired mutations could be made to serve as a check to prevent side effects on healthy cells. The recognition site might be used simply to allow correcting a single mutation, but given that the cancer process involves many events in many of the cell's compartments in many instances the mutation recognition will serve as confirmation for the vector to effect a fatal cleavage or to insert a DNA sequence designed to be fatal to the cell. In nuclear DNA success rates are expected to be higher because of the limited number of targets in each cell as compared to the multiple copies in each mitochondrion and the multiple mitochondria per cell. To further improve efficacy and to take into account the continuing change as cancers develop and mature multiple targets and/or multiple fatal outcomes can be programmed into the editing processes.

For mitochondria the sheer number is complicating. With so many copies of the genome and possibly multiple variants even in one mitochondrion, the task is significantly more complex. First, the large number of copies suggests that rather than a single copy of editing machine or group of editing machines being delivered to a designated cell, an intracellular self-replicating machine or group of machines may be more effective. Once in the cell the editing tool would co-opt the cell's machinery to proliferate inside the cell in sufficient numbers to have the desired effect(s). Unlike the difficulties presented in trying to correct a mitochondrial disease where the intent is to make multiple corrections to preserve the cell and other cells throughout the organism, generally the desired effect in cancerous cells will be fatal to the cell.

One exploitable feature is that if a significant number of mitochondria are compromised, the mitophagy/autophagy process, Ca⁺⁺ leakage, pore openings, cytochrome c release, etc. will induce or cause the targeted cell's death. Thus when the intent is to destroy rather than correct hyperproliferating cell only a portion of mitochondria need be compromised. Accordingly, one preferred strategy for triggering death of cancer cells is delivery of a proliferating mitochondrial vector to a targeted cell wherein a sufficient number of the mitochondria are modified either in the mitochondrial genome, mitochondrial membrane, delivery of components to the mitochondria, etc., to cause the mitochondria to elicit cell death.

Although bacteria are not instrumental in such gene manipulative strategies they may be selected and/or engineered to be used in conjunction with multi-pronged tactics.

Countering Anti-Apoptotic Activities

The invention may incorporate actions and/or compositions the impact transcription, translation, cytoskeleton control or other factors that modulate the propensity or ability of proteins which disfavor apoptotic events in the cell. These proteins include, but are not limited to: Bcl2, BclXI, BclxES, and Nip3.

Encouraging Pro-Apoptotic Activities

The invention may incorporate actions and/or compositions the impact transcription, translation, cytoskeleton control or other factors that modulate the propensity or ability of proteins which favor apoptotic events in the cell. These proteins include, but are not limited to: Bax, Bak, Bad, Bid, Bim, NoxA, Puma, proline oxidase, p53, cytochrome C, Hsp10, SMAC/DIABLO, apoptosis inducing Factor (AIF), endonuclease G, IAP inhibitor: omi/high temperature requirement protein A2 (HtrA2), adenine nucleotide translocator (ANT), cyclophilin D, peripheral benzodiazepine receptor, and procaspases.

Mitochondrial and Nuclear Genomes are Distinct but Work in Concert

The mitochondrial genome and the mitochondrion itself have evolved in parallel with the nuclear genome and the cells which mitochondria support. Metabolic processes (the bases of life are divided between these compartments. The mitochondrion is best known for the ETC, the TCA or Krebs cycle for efficient production of ATP. Mitochondria also are responsible for producing acetyl CoA for use in the mitochondrion and cytoplasm. And fatty acid oxidation resides in the mitochondria) matrix.

Metabolism of Fatty Acids is Shared Between mitochondrial Matrix and Cytoplasm

Shorter fatty acids can diffuse into the mitochondrion. However, longer fatty acids are reacted with coenzyme A to become esterified as a fatty acyl-CoA. This complex is carried into the intermembrane space, but must be back-converted to acyl-CoA to cross the inner mitochondrial membrane and gain access to the enzymatically active matrix.

B oxidation of fatty acids takes a long route. Free fatty acids are carried by a transporter protein e.g., FAT/CD36, SLC27, FATP, and FABPₚₘ from the interstitial space to the cytoplasm. Or fatty acids can be made available internally by autophagy or other degradative processes. In the cytoplasmic compartment the fatty acid is adenylated consuming two active phosphates (ATP4 AMP) before a CoA group is added to the fatty acid by fatty acyl-CoA synthase (FACS) to make long-chain fatty acyl-CoA. But long chain fatty acyl-CoAs cannot cross the mitochondrion's outer or inner membranes. Carnitine palmitoyltransferase 1 (CPT1) substitutes carnitine for CoA an acyl group on -Co-A to form carnitine-CoA which then crosses the outer membrane to the intermembrane space. This is repeated to cross the inner mitochondrial membrane. The transporter-carnitine translocase (CAT), exchanges long-chain acylcarnitines for carnitine molecules thus recycling carnitine for the next transport. At the inner mitochondrial membrane CPT2 then converts the long-chain acylcarnitine back to long-chain acyl-CoA.

The long-chain acyl-CoA enters the fatty acid B-oxidation pathway that produces one acetyl-CoA from each cycle of fatty acid B-oxidation. In this process each removal of acyl-CoA by acyl-CoA dehydrogenase, yields a shortened fatty acid transenoyl-CoA and one FADH₂. The transenoyl-CoA Is hydrated by enoyl-CoA hydratase to hydroxyacyl-CoA. This is reduced by NAD and B-hydroxyacyl-CoA dehydrogenase to B-ketoacyl-CoA. Acyl-CoA acetyl-transferase then adds another CoA while cleaving one-acetyl CoA. Acetyl-CoA can condense with oxaloacetate to enter the citric acid cycle as citrate. NADH and FADH2 produced by both fatty acid B-oxidation and the TCA cycle are used by the ETC to produce ATP.

A partial reverse of this process is used to produce ketone bodies especially essential to the central nervous system when glucose is unavailable. Two acetyl CoAs can be converted by thiolase to acetoacylCoA which HNG-synthase catalyzes to form HMG-CoA. Then HMG-CoA lyase forms one acetoacetate and regenerates a CoA. B-hydroxybutyrate dehydrogenase converts the acetoacetate molecules to B-hydroxybutyrate available to maintain brain activity in the absence of available glucose.
Krebs Cycle is Normally a Major mitochondrial Metabolic Emphasis

The Krebs cycle for which the mitochondrion is probably best known is summarized in the diagram below:

glucose + ATP

hexokinase

| | |
|---|---|
| glucose-6-phosphate (G6P)+ ADP | 4 ribose-5-P + |

NADPH

[pentose phosphate pathway]
phosphoglucose isomerase

fructose-6-phosphate + ATP

phosphofructokinase (PFK) (inhibitors:
phosphoenolpyruvate

(PEP), ADP

fructose-1,6-bisphosphate + ADP

aldolase

glyceraldehyde-3-phosphate + dihydroxyacetone phosphate

dihydroxyacetone phosphate triose phosphate

isomerase glyceraldehyde-3-phosphate

(2x) J,
glyceraldehyde-3-phosphate + P,+ (NAD⁺)
(NAD⁺ −> NADH)

Glyceraldehyde-3-phosphate

dehydrogenase 1,3-bisphosphoglycerate + (NADH)

1,3-bisphosphoglycerate + ADP

phosphoglycerate kinase

3-phosphoglycerate (3PG) + ATP

phosphoglycerate mutase

2-phosphoglycerate

enolase [Mg⁺⁺ cofactor]

phosphoenolpyruvate + H₂O [See PFK above]
phosphoenolpyruvate + ADP

pyruvate kinase

pyruvate

+ ATP
pyruvate +
NADH

lactate dehydrogenase A (LDH-A)

lactate + NAD⁺

Supporting the Krebs cycle and slowing the ribose pathway are examples of metabolic influences countering cancer.
Metabolic Pathways in Blood-Vessel Formation.

The formation of blood vessels depends on the proliferation and migration of endothelial cells—processes that require production of the metabolite acetyl-CoA from mitochondria. Conversion of glucose, glutamine and other nutrients into acetyl-CoA is required for the production of energy and macromolecules, both of which promote endothelial-cell migration to the metabolizing site. The interconnected metabolic pathways make the production of acetylCoA, from oxidation of fatty acids, essential for DNA synthesis and endothelial-cell and any other cell proliferation.

Vitamin B3, Niacin: In addition to its well-known redox functions in energy metabolism, niacin, in the form of NAD, participates in a wide variety of ADP-ribosylation reactions. Poly(ADP-ribose) is a negatively charged polymer synthesized, predominantly on nuclear proteins, by at least seven different enzymes. Poly(ADP-ribose) polymerase-1 (PARP-1) is a major participant in polymer syntheses and is important in DNA damage responses, including repair, maintenance of genomic stability, and signaling events for stress responses such as apoptosis. PARP-1 is therefore a prime target when metabolic modulation is in play.
G Proteins

NAD is also used in the synthesis of mono(ADP-ribose), often on G proteins. Sequencing the human and other animal genomes has made obvious the number and importance of G proteins for signal transduction, and as targets for therapeutic intervention.

Several G proteins act through stimulating production of cyclic AMP (cAMP) from ATP through stimulating the membrane-associated enzyme adenylate cyclase. cAMP then act as a second messenger that activates protein kinase A (PKA). PKA under different conditions phosphorylates many different downstream targets, including, but not limited to: anti-diuretic hormone (ADH, aka vasopressin), growth hormone releasing hormone (GHRH), growth hormone inhibiting hormone (GHIH, aka somatostatin), corticotropin releasing hormone (CRH), adrenocorticotropic hormone (ACTH), thyroid stimulating hormone (TSH), thyrotropin releasing hormone (TRH), lutinizing hormone (LH), follicle stimulating hormone (FSH), parathyroid hormone (PTH), Calcitonin, glucagon, chorionic gonadotropin (CG), and epinephrine.

Vitamin B3—Niacin

NAD and NADP are required for the synthesis of cyclic ADP-ribose and nicotinic acid adenine dinucleotide (NAADP). These compounds control intracellular calcium signaling. Modulating any of these processes has the potential to impair genomic stability which might deregulate cell division and contribute to enhanced cancer activity.
Vitamin B6—Pyridoxine

High dietary vitamin B6 attenuates and low dietary vitamin B6 increases the risk of cancer. Vitamin B6 is present in many foods so severe deficiency is uncommon. But even in the absence of a clinical deficiency availability of B6 may be sub-optimal, especially with respect to rapidly proliferating cancer cells. B6 is an important enzymatic cofactor. See, e.g., heme synthesis discussed later. Modulating B6 availability to the organism or to a cell or a group of cells in the organism can be one tool in modulating and balancing metabolism in favor of limited proliferation.
Examples of Key Nodes in Metabolic Control of the Ribose Pathway

NADPH inhibits conversion of G6P to gluconolactone. Ascorbate/Cu⁺⁺, ascorbate/Fe⁺⁺, Cu⁺⁺, diazotetrazole, and GSH are inhibitors of gluconolactonase that converts gluconolactone to 6-phospho-D-gluconate. triphenylmethane and derivatives: bromocresol purple, bromocresol purple-salt, bromochlorophenol blue-salt, bromophenol blue-salt, tetraiodophenol-sulfonephthalein-salt, ethylenesulfonic acid oligomer, 4-phospho-derythronate, 2-deoxy-6-phospho-D-gluconate, 5-phospho-D-ribonate, 6-aminonicotinamide, 6,7-dideoxy-7-phosphono-d-glucoheptonate, 6-deoxy-6-phosphono-d-gluconate, 5-phospho-dribonate, 4-Phospho-d-erythronate, each inhibit 6-phosphogluconate dehydrogenase thereby preventing additional NADPH and ribulose phosphate formation. The inhibitory salts are salts most commonly using a monovalent cation, and very often sodium salt is most available in the open market. However, other salts may be selected when the benefits justify the additional efforts required to obtain and deliver them.

Oxythiamine and p-hydroxyphenylpyruvate inhibit transketolase and arabinose-5-phosphate; and fructose 1,6-bisphosphate inhibits transaldolase. This inhibition may in some instances be counterproductive because these enzymes scavenge ribose-5-P and divert the carbon to fructose-6-P and gluceraldehyde-3-P. Ribose-5-P is used for nucleic acid synthesis, but blocking the pathway may have its advantages.
D3 Inhibition of the Hedgehog Signaling Pathway

The hedgehog (Hh) signaling pathway is a developmental pathway which plays a key role in directing growth and tissue patterning during embryonic development. Dysregulation of Hh signaling contributes to the development of a variety of tumors, including skin, brain, colon, pancreatic, and lung cancers. When constutively activated, this pathway results in the increased expression of Hh target genes, including several forms of the glioma-associated oncogene (Gli) family of signaling proteins. These events are associated with uncontrolled tumor proliferation. Research has demonstrated that the anti-proliferative activity of Hh pathway inhibitors (including, e.g., Cyc, GDC-0449, and VD3) in cultured cancer cell lines does not correlate with pathway inhibition in Hh-dependent cells. However, each of these compounds has modest anti-proliferative effect in multiple cell lines, suggesting either Hh signaling plays a role in preventing cancer cell growth in vitro or that anti-proliferative effects of these compounds are mediated through unidentified cellular mechanisms not associated with Hh inhibition.

With respect to VD3, cellular effects unrelated to Hh signaling likely result from activation of VDR signaling. Therefore, applying the anti-proliferative activity of the VD3 analogues could demonstrate ability to selectively inhibit the Hh pathway.

In several cancer cell lines, increased expression levels of vitamin D metabolizing enzymes for example in U87MG cells suggests that the enhanced anti-proliferative effects may result from the cellular conversion of VD3 to 25-hydroxy-D3 and/or to 1a,25-hydroxy-D3 and to subsequent activation of VDR.

Recent studies have demonstrated that several natural and synthetic cholesterol metabolites, including oxysterols and bile acids, present anti-proliferative effects. These considerations may be selectively used in conjunction with the bacterial strategies outlined herein in accord with a practitioner's discretion.

Cellular Metabolism and Disease

To date, more than 8,700 reactions and 16,000 metabolites are annotated in the Kyoto Encyclopedia of Genes and Genomes (see e.g., http://www.genome.jp.kegg/ pathway.html). At its base, metabolism can be simplified to pathways involving nutrients: carbohydrates, fatty acids, and amino acids, that are essential for energy homeostasis and synthetic metabolism. In most mammalian cells, growth (anabolic metabolism) occurs only when promoted by extracellular ligands. These growth factors stimulate signal transduction pathways including, but not limited to: the phosphatidylinositol 3-kinase (Pl3K)/Akt, and the mammalian target of rapamycin (mTOR) pathway. Many additional pathways and points for modulating same are discussed elsewhere in the description. Activation of Pl3K/Akt like other pathways alters the phosphorylation states of numerous target proteins (whose phosphorylation status determines activity levels) which together coordinate cellular activities including those that coordinate cell division or proliferation. But a successful transition from a resting state to growth can only occur if metabolism is adjusted to meet the rising demands for molecules like nucleic acids that are necessary precursors to a cell's proliferation.

Growth factor-induced signaling is a common practice for organisms to coordinate these functions. Underlying this is a requirement for maintaining a bioenergetic state permissive for growth. For a cancer cell to proliferate it must have previously made the macromolecules necessary for both daughter cells and must have consumed and now stored sufficient energy to accomplish the task.

In particular, the Pl3K/Akt/mTOR pathway is commonly activated in proliferating cell because it both stimulates a rapid increase in essential nutrient uptake and directs the allocation of these nutrients into catabolic and anabolic pathways needed to produce the energy and macromolecules. Interference with any of these downstream metabolic effects can render the growth factor initial stimulation ineffective.

Dynamic mechanisms also sense cellular energy status and regulate a balance between anabolism and catabolism. Whereas the Pl3K/Akt/mTOR pathway promotes anabolism and suppresses catabolism, AMP-activated protein kinase (AMPK) does the reverse. This serine-threonine kinase is a “fuel sensor” that becomes activated during a compromised bioenergetic state such as acute nutrient deprivation or hypoxia. By phosphorylating a number of gatekeeper targets, AMPK down-regulates energy-consuming, growth-promoting pathways like protein and lipid synthesis and up-regulates catabolism of fatty acids and other fuels. This enables the cell to rebalance energy supply with demand.

AMPK also regulates a p53-dependent, cell-cycle checkpoint activated by glucose deprivation thereby limiting growth when glucose supply is weak. AMPK also coordinates expression of stress response genes by migrating to chromatin and phosphorylating histone H2B on its S36. This modulating activity synergizes AMPK's effects on gene expression in the nucleus. As a result, AMPK executes and controls several activities that allow cells to respond emphatically and comprehensively to energy shortage. In mammals, cell growth and proliferation are controlled by extracellular factors that bind to receptors on the plasma membrane that include, but are not limited to: hormones, growth factors, cytokines, specific nutrients, etc. These ligands bind to cell surface receptors and initiate signal transduction cascades that stimulating numerous cellular activities to enable growth and replicative division. Appropriate control of metabolism is required for these effects to achieve valid results. For example, one of the proximal effects of growth factor signaling is to increase surface expression of transporters, for glucose and other nutrients, which when consumed provide energy and metabolic precursors to produce needed macromolecules. Catabolism of these nutrients generally ends with carbon dioxide and energy. If nutrients are present in excess so that flux through these foundational catabolic pathways is satisfied, other pathways branching from core metabolisms are induced to propagate growth signals internally and/or for export.

Hexosamine biosynthesis reinforces growth signals by enabling cells to maintain protein synthesis for example for cell surface expression of growth factor receptors and of nutrient transporters. Acetyl-CoA generated by acetyl-CoA synthetases (ACS) and ATP-citrate lyase (ACL) provides substrate needed to synthesize lipids and other macromolecules and for acetylation reactions that regulate gene expression and resultant enzyme functions. A favorable energy state during growth factor signaling also suppresses AMPK, thereby permitting cells to engage in energy-consuming biosynthetic pathways and to progress through the cell cycle.

The TCA cycle of the mitochondrion serves a biosynthetic role in addition to its more familiar function as energy deliver. Requirements of the rapidly proliferating cells for production of specific biosynthetic products would control the relative importance of the TCA cycle in tumors. Precursors for: protein, lipid, and nucleic acid synthesis are produced in the TCA cycle. Export of these precursors from the cycle to supply macromolecular synthesis is a prominent feature of proliferating cancer cells. Pyruvate carboxylation is one of several mechanisms by which carbon can be resupplied to the TCA cycle to offset precursor export. Such processes, termed anaplerotic pathways, prevent TCA cycle intermediates from becoming detrimentally depleted during cell growth.

18F-fluorodeoxyglucose-PET (FDG-PET), which is commonly used to assess lung tumors, can identify localized areas of intense glucose uptake. Multiparametric MRI would also be particularly useful for this type of analysis, since it can assess regional heterogeneity of perfusion, oxygenation, cellularity, necrosis, temperature, and other characteristics relevant to cancer cell metabolism.

Cells have two ways to produce adenosine triphosphate (ATP) for energy: glycolysis and oxidative phosphorylation (OXPHOS). In glycolysis, glucose is converted to pyruvate, while generating NADH from NAD⁺ and ATP from ADP and inorganic phosphate. If the pyruvate is reduced to lactate, NAD⁺ is regenerated and glycolysis continues. Although glycolysis is rapid, it is deemed inefficient because most of the energy that could be generated from glucose is lost when the cell secretes lactate, a three carbon molecule retaining significant energy in its bonding structure. In contrast, OXPHOS is highly efficient about 20-fold more efficient per ATP molecule generated. When substrates like pyruvate are oxidized in the mitochondria, reducing equivalents are provided to the ETC, creating a proton gradient that drives ATP synthesis. The vast majority of cancer cells use both glycolysis and OXPHOS together to satisfy metabolic needs, although OXPHOS I reduced in its importance as mitochondria are co-opted for supporting essential proliferation pathways, the balance between the two can vary widely in different cancer types and at different phases of cancer development.

Genes involved in the rebalancing relate to a large number of the cells pathways and their enzymes, including, but not limited to: STAT1, Akt, Jak/Tyk2, CUG2, HGPRT, SETDB1, LDH1, etc.

Especially notable or pathways leading to lactic acid formation, thus sparing mitochondrial activity from having to metabolize pyruvate, pathways leading to purine and pyrimidine manufacture to support nucleic acid synthesis, pathways leading to angiogenesis, pathways sparing the proliferating cell from cell death or apoptosis, pathways that may be activated to drive the proliferating cell towards cell death or apoptosis and pathways that control cell division and cell cycle.

A low-molecular-weight compound secreted appears responsible for enhanced CLL cell survival. This compound is probably the amino acid cysteine, one of the three amino acids required to synthesize glutathione, and protector against oxidative damage and maintenance of volume in tissues such as the cornea. Most cells, including cancer cells, do not rely on extracellular cysteine for their glutathione biosynthesis. Rather, cells take up the more abundant and more stable oxidized form, cystine (two cysteine molecules joined by S—S bonding). Cystine is readily reduced to cysteine for synthesizing polypeptides inside the cell. Cystine is taken up through the Xcantiporter, a multimeric amino-acid transporter that exchanges glutamate for cystine at the cell surface.

Hypoxic tumor cells appear to favor conversion of glucose to lactate, which is disposed of by secretion (or export) into the interstitial fluid (extracellular compartment) where it can be metabolized by cells in areas of more abundant oxygen—either because of better vascularization of because of lower metabolic demands. In breast cancer, tumor cells derived from the luminal epithelium synthesize glutamine de novo before secreting it. So while controlling glutamine availability may be an important support in methods of the present invention the same method(s) will not apply to all instances where the invention is practiced. Obviously these luminal epithelial derived cancer cells and other cancer cells with similar metabolic modifications can thrive under conditions of glutamine deprivation.

By contrast, cells derived from the basal epithelium do not synthesize glutamine and therefore require an extracellular source. These cells can be rescued by co-culturing them with glutamine-secreting luminal cells, raising the possibility of regional heterogeneity in glutamine dependence in normal and tumor-tissue.

One mechanism evidencing benefits of controlling glutamine access is provided by understanding the mitochondrial enzyme glutaminase C (GAC) which catalyzes the hydrolysis of glutamine to glutamate plus ammonia (NH₄⁺). Such glutamine focused diversion appears to be a key step in the modifications of metabolism, in general, and of glutamine, in particular, by cancer cells. Because glutamine is necessary for a range of biochemical reactions, notably including nucleotide and protein synthesis, glutamine analogs like the GLS1 inhibitor diazo-O-norleucine (DON) may not be ideal candidates for cancer drugs as a broad class.

However, two classes of allosteric GAC inhibitors have been identified and may present more promise as active ingredient compounds for cancer therapeutics. One of these inhibitor groups consists of analogs of bis-2-(5-phenylacetamido-1,2,4-thiadiazol-2-yl)ethyl sulfide (BPTES), a reversible GAC inhibitor. X-ray crystal structures of the GAC-BPTES complex show that BPTES effectively traps GAC as an inactive tetramer. A second, more recently identified, class of allosteric GAC inhibitors, a class that is highly specific for inhibiting cancer cell growth while having little effect on normal (nontransformed) cells is represented by the benzophenanthridinone 968. Oncogenic Dbl induction did not cause marked increases in glucose-fueled anaplerosis, as measured by ¹³C enrichment in citrate, when using [U-¹³C]glucose as a tracer, demonstrating that a highly specific stimulation of glutamine metabolism accompanies Rho GTPase-dependent transformation.

Reductive Carboxylation Supports Growth in Tumor Cells with Defective Mitochondria

Mitochondrial metabolism provides precursors for macromolecules in growing cancer cells. In normally functioning tumor cell mitochondria, the oxidative metabolism of glucose-derived and glutamine-derived carbon produces citrate and acetyl-coenzyme A for lipid synthesis, an important activity to support tumorigenesis. And some tumors bear mutations in the citric acid cycle (CAC) or electron transport chain (ETC) that disable normal oxidative mitochondrial function. Yet in a large number of tumors, the citric acid cycle and the ETC remain functionally intact, though de-emphasized in activity. It is not understood how cells from CAC and/or ETC deficient tumors generate precursors necessary for macromolecular synthesin support of proliferation. But cells with defective mitochondria likely use glutamine-dependent reductive carboxylation rather than oxidative metabolism as the major pathway of citrate formation. This pathway uses mitochondrial and cytosolic isoforms of NADP1/NADPH-dependent isocitrate dehydrogenase, and subsequent metabolism of glutamine-derived citrate provides both the acetylcoenzyme A for lipid synthesis and the four-carbon intermediates needed to produce the remaining CAC metabolites and related macromolecular precursors. Cells with intact CAC and ETC may down-regulate these paths to better support the synthesis of purines, pyrimidines and other macromolecule precursors. This is probably a strong hypothesis given that reductive, glutamine-dependent pathway is the dominant mode of metabolism in rapidly growing malignant cells containing mutations in complex I or complex III of the ETC, in patient-derived renal carcinoma cells with mutations in fumarate hydratase, and in cells with normal mitochondria subjected to pharmacological ETC inhibition.

Induction of a versatile glutamine-dependent pathway that reverses many of the reactions of CAC supports tumor cell growth, and illustrates how cells may generate satisfactory pools of CAC intermediates in the face of impaired mitochondrial metabolism.

As expected for cells with defective oxidative phosphorylation, a model cell line, CYTB 143B cells, had higher glucose consumption and lactate production than WT143B cells, demonstrating the metabolic shift towards aerobic glycolysis.
Glutamine and Cancer: Cell Biology, Physiology, Clinical Strategies

In mitochondria, under the influence of Hifla, pyruvate dehydrogenase kinase (PDH), (PDK) blocks the activation of mitochondrial pyruvate dehydrogenase thereby limiting the pyruvate conversion into acetyl-CoA. Hifla (hypoxia inducible factor la) also stimulates expression of LDH-A to generate NAD⁺. LDH-A hyperactivity appears essential for scavenging pyruvate to maintain NAD⁺ and/or to remove pyruvate stimulus of the mitochondrial pyruvate to acetyl-CoA Krebs mission. But Krebs is still able to partially cycle when glutamine is deaminated to glutamate in a reaction supporting synthesis of the pyrimidines and purines used for nucleic acids. The glutamate enters the mitochondrion as a-ketoglutarate which progresses through maleate, exits the mitochondrion then is converted to pyruvate and lactate.

Protein kinase B (PKB, aka Akt) is important for regulating the glycolytic over OXPHOS favoritism. PKB/Akt after being phosphorylated by phosphatidylinositol 3 kinase (P13K) takes residence in the plasma membrane and inhibits or slows several paths such as increased cAMP response element binding protein (CREB) with actions impacting Alzheimer's, spatial and long term memory, c-fos, tyrosine hydroxylase, time keeper genes—Period1 and Period2 (PER 1 and 2), and many other important neuropeptides such as somatostatin. The target of CREB is the sequence TGACGTCA which will be left unhindered when it benefits from C methylation. CREB also influences the plasma membrane though its activation of P13K which controls positioning and polarity of receptors in plasma membranes. P13K activation is essential in forestalling differentiation in favor of proliferation and thereby plays a key role in supporting cancer proliferation and slowing apoptosis. Stimulation by insulin, insulin-like growth factor 1 (ILGF1 or an alternate name somatomedin C), calmodulin, epidermal growth factor, sonic hedge-hog, and the like, favors “proliferation and growth” over functional differentiation and culling (apoptosis).

Homeobox 9 (HB9), phosphatidylinositol-3,4,5-trisphosphate 3-phosphatase (PTEN), glycogen synthase kinase 3 β (GSK3B)

The diversion of G6P to the R5P pathway, to generate ribose-5-phosphate and NADPH, supports the enhanced nucleotide synthesis necessary for transcription and translation. The NADPH is also a major anti-oxidant and participant in bio-synthesis. The 3PG is a reactant for both amino acid and fatty acid and other synthesis.

3PG is notable for its conversion to serine which serves as a carbon source for the folate cycle through its conversion of tetrahydrofolate (THF) to methyl-tetrahydrofolate (mTHF). NADPH is oxidized as part of the folate cycle. MCT4 is necessary for removing the lactate from cell's cytoplasmic space. Since the lactate production is enhanced in proliferating cancer cells, especially growing, vascularization deprived, and/or hypoxic cancer cells, interference with formation of intact functioning transporter including, but not limited to stopping or altering: transcription, translation, expressing, processing, transport to or insertion in plasma membrane and maintenance within the membrane will seriously compromise cell survival.

A seemingly opposite strategy can augment or synergize this result. Since neighboring cells, especially neighboring well-oxygenated cancer cells, may remove lactate from interstitial space and cycle it though lactate dehydrogenase for metabolic use or may otherwise remove lactate, by blocking or slowing MCT1, the relevant lactate uptake transporter, a toxic buildup of lactate in the interstitial space which then would back up into cells to can promote necrosis or apoptosis of these cells.

In some tumor situations, blocking lactate uptake can severely increase the demand for glucose, which when unmet starves the cells into a necrotic or apoptotic, or extreme quiescent state. Glycine also is involved in the folate cycle. So stopping glycine C-transferase activity and/or glycine dehydrogenase activity phosphoaminotransferase (PSAT) and/or serinehydroxymethlase (SHMT) takes away these pathways for tumorigenesis. Especially in colon cancers stimulating fragile histidine triad may maintain genome stability, but its absence is compatible with rapid mutation as observed in cancer cells.

Activation of the Pl3K-Akt pathway will increase glucose uptake and metabolism because Akt phosphorylates and inactivates FOXO. This down regulates PGC1a and inhibits mitochondrial biogenesis. When MYC is activated glutaminolysis is induced—glutamine is converted to a-ketoglutarate (aKG). Then reductive carboxylation of aKG using NADPH-linked IDH2 results in isocitrate and more citrate available for export to the cytosol, where isocitrate is available for conversion back to aKG by NADr-linked IDH1. Or citrate may be exported from the mitochondrial matrix to the cytosol where it is cleaved by ATP citrate lyase (ACL) to produce oxaloacetate (OAA) and acetyl-CoA.
Diverse Functions if Glutamine in Metabolism, Cell Biology and Cancer

Glutamine is an amino acid, one of the constituents of proteins. Glutamine, a carbon rich molecule, is also an acceptable substitute for glucose as the cell's fuel. The ready alternatives available as substrate for various metabolic functions and alternative pathways available to achieve the necessary functions suggest two main approaches for external control of unwanted cell growth and proliferation.

A first approach would be to block metabolism at an initiation step critical to many downstream paths or to block a junction point critical to several alternative path.

A second approach would be to therapeutically manipulate several interfacing or parallel paths. Glutamine because it can participate in many functions, including, but not limited to: a carbon source for building biomolecules, an energy source for generating needed ATP, and a conduit of nitrogen between cells and parts of cells. Glutamine with all its use is not surprisingly the most common amino acid (about ⅕ of the amino acids) free in circulating blood. Glutamine, although capable of being synthesized in mammalian cells, often is in short supply for all the metabolic demands it can satisfy. Glutamine is exported to circulation as a non-toxic carrier of NH₄⁺ for example from breakdown of other amino acids. Glutamine is a major source of urea, the chemical carrier of nitrogen out of the body in renal waste. As a nutrient for cancer cells glutamine is often, but not always, available from the circulatory system.

Another prime source of glutamine is proteins as they are recycled during normal metabolic processes. The extraordinary consumption of glutamine in cancer cells is evident in the activity of oncogenic RAS to stimulate macropinocytosis, a process through which extracellular molecules, e.g., proteins are ingested by the cell in the form of macropinosome vesicles. These vesicles can merge to intracellular lysosomes for degradation of the engulfed proteins to useful building blocks. Amplifying this macropinosomic lysosomic activity by internal or external signaling paths, like amplifying other lysosomic activities is one means of initiating apoptotic cell death. Mitochondria participate in glutamine recycling through several aminotransferases discussed below. A glutamine transport protein e.g., SLC1A5 internalizes circulating glutamine. In the cytoplasm, glutamine can be converted to nucleotides and uridine diphosphate N-acetylglucosamine (UDP-GlcNAc). Nucleotides are essential molecules for making genes in dividing cells.

N-glycosylation serves to stabilize proteins by maintaining appropriate 3D folded structure and to package for secretion to extracellular space. Alternatively, glutamine can be converted to glutamate by glutaminase (GLS or GLS2). The glutamate may be used to generate glutathione (an anti-oxidant protectant) or may be processed into other metabolic substrates, such as a-ketoglutarate (a-KG). The importance of this path is emphasized by the parallel pathways, i.) glutamate dehydrogenase GLUD which comes in two forms, GLUD1 and GLUD2, and ii.) aminotransferases. GLUD is activated by ADP and inhibited by GTP, palmitoyl-CoA and SIRT4-dependent ADP ribosylation. Leucine by itself allosterically activates GLUD and by acting through mTOR suppresses SIRT4 expression thereby accentuating GLUD activity even more. When ADP levels increase e.g., by consumption of ATP in excess of creation, this may operate as a signal for GLUD to increase its ATP output. GLUD has NH₄⁺ as a product which might be detoxified by conversion to glutamine! Whereas the aminotransferase path used to make other amino acids, aminotransferase reactions can occur both in the mitochondria and in the cytoplasm. In some tumors 50% or more of the non-essential amino acids used to build proteins are derived from glutamine. And glutamine through its involvement in aspartate synthesis is a key element for making the purines and pyrimidines necessary for nucleic acids. Then in the mitochondrion a-KG can participate in the tricarboxylic acid (TCA) cycle through succinate and fumarate to malate thereby providing ATP for the cell.

Malate can leave the TCA cycle to produce pyruvate and NADPH. Remaining in the mitochondrion, malate cycles to oxaloacetate (OAA), which may leave the cycle as aspartate to support nucleotide synthesis, e.g., DNA or tRNA for a dividing or rapidly metabolizing cell. As another option available in the cell's metabolism, a-KG can reverse through the TCA cycle, in a process called reductive carboxylation (RC) to form citrate, to make acetyl-CoA and lipids. The requirements of tRNA (and probably to a lesser degree, mRNA) and DNA for the growing and proliferating cell are perhaps the most likely rational for a cancer cell's metabolic shift in favor of glutamine.

Two glutaminase enzymes (GLS and GLS2) are differentially expressed depending on tissue type. GLS which has two alternative splice forms (GLC and KGA) is activated by phosphorylation, but receives feedback inhibition by its glutamate product. GLS2 however increases activity as its NH₄+increases abundance. These enzymes are regulated by sirtuin 5 (SIRTS) which down-regulates GLS and SIRT3 which up-regulates GLS2 (especially during times where caloric intake is wanting). pH is an important modulator of GLS mRNA and its expression and activity can be controlled at the site of transcription (in the nucleus), and later in cytoplasmic environment by microRNAs and RNA binding proteins directing mRNA processing and alternative splicing. Splice variant GAC appears more prevalent in many cancers and is the more active variant. The cell's favoring of this variant would not be apparent in a nuclear genome sequencing, but might be seen in a complete sequence analysis that also monitors expression. GLS2 can be turned off by methylation which has been observed in some cancers, especially hepatic forms. GLS2 methylation may also be important for cancer cell creation in that this enzyme may have another quality or side effect in its propensity to bind RAC1 cutting metastasis.

The aminotransferase family includes several forms. Better characterized family members include alanine aminotransferase (aka glutamate-pyruvate transaminase), aspartate aminotransferase and phosphoserine aminotransferase (PSAT1). Alanine aminotransferase comes in a mitochondrial isoform GPT2 and a cytoplasmic isoform, GPT. Similarly, aspartate aminotransferase has a cytoplasmic isoform, GOT1 and a mitochondrial isoform, GOT2. PAT1 appears to be preferentially expressed in tumor cells and thus controlling its activity can be one tool for stressing cancer cells. In cancer cells where hypoxia-inducible-factor-a (Hifa) is constitutively expressed or where mitochondrial participation in fatty acid synthesis is severely compromised, glutamine may see further use in reductive carboxylation to synthesize fats.

Glutamine metabolism is crucial for cellular reactive oxygen species (ROS) homeostasis. Glutathione (GSG), one of the important reactive oxygen scavengers, requires glutamine as a raw material for the amino acid components of GSG. Many studies have shown that glutamine availability is rate limiting in GSG synthesis. ROS effects are complicated. Under some conditions increased ROS (a sign of cell stress) initiates apoptosis. But some cancers as part of their development process have survived by downplaying the apoptotic input of increased ROS. In these cases ROS can cause internal oxidative damage within the cells. Glutamine also is involved in the TOR pathway. TOR encourages growth and inhibits autophagy. Glutamine suppresses pro-apoptotic action of GCN2 and Integrated Stress Response (ISR).

Oncogenic genes upregulate glutamine uptake and metabolism as observed in the Q (glutamine) metabolism stimulated by HIF2 and MYC. When glutamine is metabolized, its carbon mass is preserved chiefly in amino acids and fats while the nitrogen is an integral component for nucleic acid synthesis. Through aspartate transamination glutamine can also contribute carbon atoms to purines and pyrimidines of the nucleic acids. Glutamine can serve an intracellular signal through mTOR to activate carbamoyl-phosphate synthetase 2, aspartate transcarbamylase, and dihydroorotase (CAD), responsible for using nitrogen from glutamine to synthesize pyrimidine. Although tumor cells tend to have large intracellular pools of glutamate, maintaining these pools rests on the ability to convert glutamine into glutamate because glutamine is an abundant extracellular nutrient and glutamate is not. This process is largely because of the activity of phosphate-dependent glutaminase (GLS), a mitochondrial enzyme that is highly expressed in tumors and tumor cell lines.

Classical experiments have shown that GLS activity correlates with tumor growth rates in vivo, (Knox et al. 1969; Linder-Horowitz et al., 1969), and experimental models to limit GLS activity resulted in decreased growth rates of tumor cells and xenografts.

The rate-limiting step in the formation of hexosamine is catalyzed by glutamine:fructose-6-phosphate amidotransferase, which transfers glutamine's amido group to fructose-6-phosphate to form glucosamine-6-phosphate, a precursor for N-linked and 0-linked glycosylation reactions. These reactions are necessary to modify proteins and lipids for their participation in signal transduction, trafficking/secretion and other processes. Impairment of glucosamine-6-phosphate production is thus expected to reduce cell growth and to interfere with cell signaling. Surprisingly, glutamine:fructose-6-phosphate amidotransferase activity can be suppressed by expressing an antisense GLS complementary DNA in some breast cancer cells. The disturbances of 0-linked glycosylation pathways, alters glycosylation of the transcription factor Sp-1 and increases its transcriptional activity. Glutathione (GSH) is the major thiol containing endogenous antioxidant and serves as a redox buffer against various sources of oxidative stress. In tumors, maintaining a supply of GSH is critical for cell survival because it allows cells to resist the oxidative stress associated with rapid metabolism.

GSH is a tripeptide of glutamate, cysteine and glycine and its formation is highly dependent on glutamine. Not only does glutamine metabolism produce glutamate, but the glutamate, pool is also necessary for cells to acquire cysteine, the frequent limiting reagent for GSH production. Glutaminase activity generates free ammonia, a potentially toxic metabolite. Without a mechanism to dispose of ammonia rapidly, intracellular ammonia concentrations would reach several hundred mmol/l within a few hours which would be expectedly toxic to most cells in the area. It is not understood how tumor cells dispose of ammonia during rapid glutamine catabolism. The traditional view held that passive diffusion of the gaseous form (NH₃) across the lipid bilayer accounted for essentially all ammonia transport. This simple model does not provide adequate explanation for some tissues with a high demand for ammonia transport. For example, in the kidney, in which ammonia metabolism is a key mediator of acid-base homeostasis, a number of protein transporters exist to traffic ammonia, as NH₃ and/or NH₄⁺. These systems include ion channels, aquaporins and Rh glycoproteins, some of which are overexpressed in tumors. Although the exact mechanism of tumor cell ammonia secretion has not been proven, the process bears therapeutic potential. Blocking ammonia secretion would, presumably either suppress net glutamine consumption or cause toxic intracellular accumulation of ammonia, both of which should impair cell survival and growth.

Other reports have identified a role for glutamine in extracellular signal-regulated protein kinase (ERK) signaling pathways. This phenomenon has been best characterized in intestinal epithelial cells, which consume glutamine as their major bioenergetic substrate and require glutamine for both proliferation and survival. Addition of glutamine is adequate for stimulating ERK signaling within a few minutes in porcine intestinal epithelial cells, and it enhanced ³H-thymidine incorporation. In rat intestinal epithelial cells, glutamine was shown to be comparable to serum in preventing apoptosis, and it stimulated a sustained activation of ERK signaling. The importance of glutamine as a supporter of tumorigenic activity should not be downplayed.

Inhibitors of the ERK pathway have eliminated the protective effect of glutamine supplementation. It was not clear from these studies whether glutamine import alone was required for the effects, or whether the cells needed to metabolize glutamine to activate ERK signaling. Consistent with glutamine's effects on cell signaling, a number of reports have shown that it also influences gene expression. In cell lines, addition of glutamine increases expression of the pro-proliferation factors c-jun and c-myc within a few minutes and promotes cell survival through the negative effects on growth-inhibitory and pro-apoptotic factors such as CHOP, GADD45, Fas and ATF5. In Ehrich ascites tumor cells, GLS knockdown led to enhanced phosphorylation, DNA binding and transcriptional activity of Sot. In HepG2 hepatoma cells, glutamine was required for the induction of manganese superoxide dismutase expression that accompanied the depletion of essential amino acids.

Glutamine's involvement in manganese superoxide dismutase expression was blocked by inhibiting the TCA cycle, ERK1/2 or mTOR, suggesting that an integration between mitochondrial glutamine metabolism and signal transduction facilitates the effect. Evidence shows that glutamine also modulates immune responses, though it is unclear exactly through which mechanistic paths these changes are achieved. Conceivably, glutamine could exert its effects through redox homeostasis, bioenergetics, nitrogen balance or other functions. During radiation-induced oxidative stress in the rat abdomen, pre-treatment of the animals with glutamine significantly decreased tissue inflammation and expression of nuclear factor-kB. So glutamine may be available to buffer the redox cell's capacity.

Nuclear factor-kB likely is a key mediator that links glutamine availability with stress responses, since there is an inverse correlation between glutamine abundance and nuclear factor-kB-mediated gene expression.

The role of glutamine as an immunomodulator in cancer appears promising in that the avid consumption of glutamine by tumors reduces glutamine availability for neighboring cells, and can modulate local nuclear factor-kB signaling and expression of inflammatory mediators in the stroma. Because tumor cells are exposed to many nutrients simultaneously, achieving a comprehensive view of tumor metabolism requires an understanding of how cells relate these pathways into an over-arching metabolic phenotype. For different tumor cell types and for different tumors pathway emphases would most likely vary. It is expected that the skilled artisan in practicing this invention to its best advantages would investigate glutamine effects, either by assay or trial and error or a combination thereof.

Consequently, considerations relating to glutamine should not ignore the rapid glucose utilization that often accompanies cell proliferation. The rates of glucose and glutamine consumption in general far outpace the utilization of other nutrients available to the cell. Presumably, this modified metabolism supports both bioenergetics and the production of biomacromolecule precursor pools while sparing other energy-rich substrates, such as fatty, acids and essential amino acids, for their direct incorporation into the biomacromolecules. Increased glucose breakdown provides building blocks for the synthesis of nucleotides (via glucosamine and the pentose phosphate pathway) and amino and fatty acids (from intermediates formed in the glycolytic and tricarboxylic acid cycles). In addition, local acidification of the tumor microenvironment may facilitate tumor invasion. The enhanced activity of the pentose phosphate shunt may lead to an elevated production of NADPH and glutathione (GSH) (which would increase the resistance of tumor cells against oxidative insults and some chemotherapeutic agents). Modulating the pentose phosphate pathways is one process for compromising cancer metabolism.

The non-oxidative phase involving ribulose-5 phosphate as a substrate involves catalytic activities from enzymes including, but not limited to: ribose-5-phosphate isomerase, ribulose-5-phosphate-3-epimerase, transaldolase, transketolase, etc.

Heme Synthesis—Cooperation Between mitochondrial Matrix and the Cytoplasm

Herne, an iron chelate protein, exemplifies cooperation between cytoplasmic and mitochondrial metabolisms. Herne proteins are found in all cells. The heme group I common where oxygen is found. While probably most known for the heme presence in hemoglobin and myoglobin, heme is also a component of metaloproteins such as cytochromes, including, but not limited to: p450, b-245, c, d, f, etc. Catalase is an important protector inside most cells, protecting biomolecules from ROS damage, e.g., from hydrogen peroxide (H₂O₂). Catalase sports four heme groups and thus has an involved synthesis pathway that is amenable to modulation.

Thus heme synthesis is an important component of cell's ROS defenses. And modulation of heme synthesis (several suggestions below) is a tool available for stimulating necrosis and/or a poptosis.

Herne synthesis is started in the mitochondrion where glycine, brought into the mitochondrial matrix by SLC25A and succinyl-CoA, react to form a-amino-B-ketoadipate in the presence of pyridoxal phosphate (vitamin B6) as a cofactor for the d-aminolevulinate synthase (ALAS) enzyme which then decarboxylates the complex to form d-aminolevulinic acid (ALA). CLPX acts as a chaperone to coordinate association of B6 with ALAS thus stabilizing and activating the complex.

Nutritional deficiency of vitamin B6 can limit this reaction and thus heme synthesis. The d-aminolevulinate synthase enzyme is not constitutively expressed and has a short half-life. Expression of the enzyme is induced in the presence of barbiturates and steroids such as testosterone and oral contraceptives that sport a 4,5 double bond that is accessible to 5-β reductase which itself is induced during puberty. Expression of d-aminolevulinate synthase is inhibited by negative feedback from heme and by hematin.

ALA then is transported to the cytoplasm where d-aminolevulinic acid hydratase (aka porphobilinogen synthase) condenses two ALA molecules to synthesize porphobilinogen. Zn⁺⁺ is a cofactor for this enzyme. But Pb⁺⁺ has high affinity and can displace Zn⁺⁺ and inactivate this enzyme. Lead poisoning effect on this enzyme results in increased ALA in cells and blood. Since ALA cannot progress to eventual heme synthesis there is no heme feedback to suppress ALA synthesis. ALA is a neurotoxin possibly because of the ROS it creates and possibly because it mimics the neurotransmitter, γ-aminobutyric acid.

Four porphobilinogen molecules are condensed by uroporphyrinogen I synthase to form a linear tetrapyrrole which can isomerize non-enzymatically into uroporphyrinogen I or enzymatically with uroporphyrinogen III cosynthase into uroporphyrinogen III. Uroporphyrinogen III is a substrate for vitamin B12 synthesis and chlorophyll synthesis as a branch off this heme synthesis pathway.

Uroporphyrinogen decarboxylase decarboxylates acetic groups of both uroporphyrinogen I and uroporphyrinogen III changing these groups to methyl groups and forming coproporphyrinogen I and coproporphyrinogen III, respectively. The fate of coproporphyrinogen I in the cell is unknown and may be a dead end synthetic product.

Coproporphyrinogen III then migrates back into a mitochondrion through an ATP dependent carrier ABCB6 and is oxidized by coproporphyrinogen III oxidase to form protoporphyrinogen IX.

Protoporphyrinogen IX oxidase aromatizes the ring by converting methylene bridges of protoporphyrinogen IX to methenyl bridges in protoporyrin IX. The resonance bonding improves stability of the molecule.

Ferrochelatase (FECH) then adds Fe⁺⁺ to protoporphyrin IX while reducing ascorbic acid (vitamin C) and cysteine and releasing two H. Lead which inhibited ALA also inhibits ferrochelatase.

Iron is made available to FECH in the mitochondria though a transmembrane carrier, SLC25A37 stabilized with ABCB10 bound to FECH. Then finally the HEME is exported to the nucleus through FLVCR1b for cytosolic incorporation of heme into metaloproteins.

While the Organism Cannot Survive Without Surviving Cells, Survival of Each Cell May Not Optimize Survival of the Organism

Cancer cells often upregulate the rate-limiting processes and enzymes of glycolysis, including glucose transporters, for instance as a result of the constitutive signaling through the Akt pathway or as a result of the expression of oncogenes including Ras, Src or Bcl-Abl. Failure to adapt these behaviors would be incompatible with the cell's survival. So, only cells effectively navigating these changes will survive to be observed. But since all living things in their creation have a built in drive to survive, when cell's begin to be stressed in a cancer leaning direction, the cell's evolved defense will kick in to preserve life of the cell but may not support survival strategies of the organism.

Cancer cells can accumulate defects in the mitochondrial genome, leading to deficient mitochondrial respiration and ATP generation. In some cases, mitochondrial germline mutations have been shown to provide a genetic predisposition to cancer development. This would be expected because all metabolic defects or changes can be expected to stimulate compensatory reactions which will induce further compensations, etc., within the cell.

In most cases, however, historically, such mutations are acquired during or after oncogenesis. It appears that acquired mutations in mitochondrial DNA fall into two classes. A first category includes severe mutations that inhibit oxidative phosphorylation, increase the production of reactive oxygen species (ROS) and promote tumor cell proliferation. Milder mutations could permit tumors to adapt to new microenvironments, especially when tumors progress and metastasize.

Cancer cells may adapt to decreased oxygen tension (hypoxia) that is characteristic of most, if not all solid tumors as the pre-malignant lesion grows progressively further from the blood supply. In this case, the adaptation to hypoxia would be to durably shut down mitochondrial respiration and to switch on glycolytic metabolism.

In luckily specific cases, mitochondrial enzymes can act as tumor-suppressor proteins whose mutation indirectly engenders aerobic glycolysis. The inactivating mutation of mitochondrion-specific proteins such as succinate dehydrogenase (SDH subunits B, C or D) and fumarate dehydrogenase is an oncogenic event, causing phaeochromocytoma (in the case of SDH mutations) and leiomyoma, leiomyosarcoma or renal carcinoma (in the case of fumarate dehydrogenase mutations). The loss of function of succinate or fumarate deyhdrogenases results in the accumulation of fumarate and succinate in the cytosol, respectively. This, in turn, favors the activation of the transcription factor hypoxia-inducible factor (HIF) and generates a pseudohypoxic state accompanied by HIF-dependent reprogramming of the metabolism towards aerobic glycolysis.

It is conceivable to inhibit glycolysis either by targeting glycolytic enzymes or by attempting to release hexokinase from its mitochondrial receptor, VD). Inhibitors of glycolytic enzymes that have been successfully used to slow down the growth in tumors transplanted to mice include 3-bromopyruvate (an inhibitor of hexokinase) and oxythiamine (an inhibitor of the transketolase-like enzyme).

Some glycolytic inhibitors are already being evaluated in clinical trials. This applies to 2-deoxyglucose (an inhibitor of the initial steps of glycolysis) as well as to lonidamine (TH-070), an inhibitor of glycolysis that also has direct pro-apoptotic properties.

IL-2 amplifies the body's immune system, while the TGF-β inhibitor lessens the cancer cells' ability to evade the immune system. Nano devices underdevelopment might be used to administer these or other therapeutic compounds to relevant (diseased) locations. These novel nano devices, mentioned but not required to practice the present invention can in “smart” form be outfitted with sensors and brakes for attachment or movement stoppage to at that location deliver the ported therapeutic or they may remain as marker targets for a second porter to deliver one or more therapeutics to the relevant site.

Some or all of these biological paths may be helpful in explaining how any individual specific bacterial based strategy may be operating within a specific cancer cell. But understanding the complexities of any specific cancer is not a necessity when the commonalities such as increased local temperature and decreased pH are the relevant cancer markers and drivers of the cell-based therapies. These methods while addressed as cellular intervention approaches are most relevant and applicable within a larger organism such as a human, domesticated animals, etc.

In an especially elegant version, these nanosensors are equipped with simple diagnostic tools and can be queried to report efficacy of any treatments in their vicinity. A nanogel delivery system can be used for multiple therapeutics or therapeutic combinations.

Controlling Proliferation.

The cell cycle consists of a state of quiescence (Go), a first gap phase (G1), the DNA synthesis (S phase) a second gap phase (G2), then mitosis (M), the actual cell division phase. Retinablastoma protein phosphorylation by a CDK/cyclin complex allows release of transcription factor E2F that can activate several genes including, but not limited to: cyclins A, D and E. CIP/KIP family members p21CIP1, p27KIP1 and p57KIP2 assist CDK/cyclin association. p53 regulates p21CIP1. p16INK4a and p14ARF are tumor suppressors (encoded by the same gene in overlapping reading frames)!! p16INK4a is inactivated in many cancers. p14ARF can maintain cycle arrest in G1 or G2. It complexes with MDM2 to prevent MDM2 from neutralizing p53 thereby transcriptionally activating cyclin-dependent kinase inhibitor 1A or may induce apoptosis. Hyperexpression of cyclins is one hallmark of cancer.

All patents and patent applications referenced herein are hereby in their entireties incorporated by reference.

What is claimed is:

1. A method of selectively destroying abnormal cells in a multi-cellular organism, said method comprising: identifying cells whose metabolism results in a local temperature increase and a local pH decrease, binding bacteria to said cells, said bacteria integrating within the cytoplasm of said identified cells, said bacteria proliferating within said cells with destructive results to said abnormal cells.

2. The method of claim 1 wherein said multi-cellular organism is a domesticated animal.

3. The method of claim 2 wherein said domesticated animal is selected from the group consisting of: mammals and avians.

4. The method of claim 3 wherein said domesticated animal comprises a pet.

5. The method of claim 4 wherein said pet is selected from the group consisting of: canines, felines and birds.

6. The method of claim 2 wherein said domesticated animal comprises a zoo animal.

7. The method of claim 2 wherein said bacterium proliferates within said aberrant cell resulting in aberrant cell lysis and release of a population of additional bacteria capable of selectively destroying additional abnormal cells.

8. The method of claim 2 wherein said destructive results comprise intracellular immunity.

9. The method of claim 8 wherein said intracellular immunity comprises cytochrome c release from mitochondria.

10. The method of claim 8 wherein said intracellular immunity comprises initiating apoptosis.

11. The method of claim 2 wherein said destructive results comprise tagging the plasma membrane of said aberrant cells to foment a systemic immune response.

12. The method of claim 11 wherein said systemic immune response comprises a humoral response.

13. The method of claim 11 wherein said systemic immune response comprises a cellular immunity response.

14. The method of claim 2 further comprising binging to a monocarboxylate transporter 4 protein (MCT4) on surfaces of said aberrant cells.

15. The method of claim 2 wherein said bacteria are selected from the group consisting of facultative intracellular bacteria.

16. The method of claim 15 wherein said facultative intracellular bacteria are proliferated in culture without requiring eukaryotic cells.

17. The method of claim 15 wherein said facultative bacteria are selected from the group consisting of: bartonella henselae, brucella, francisella tularensis, legionella, listeria monocytogenes, salmonella typhi, mycobacterium, nocardia, rhodococcus equi and Yersinia.

18. The method of claim 2 wherein said bacteria are cultured in eukarotic cells.

19. The method of claim 18 wherein said bacteria are selected from the group consisting of: chlamydia, coxiella and rickettsia.

20. A system of treating a cancer in a mammal, said system comprising applying the method of claim 1 within a body of a domesticated animal.

21. The system of claim 20 wherein said applying is systemic.

22. The system of claim 20 wherein said applying is selective to a region or tissue of said body.

23. The system of claim 22 wherein said region or tissue is selected from the group consisting of: endothelial, hepatic, renal, optical, nervous, pulmonary, digestive, structural and integumentary elements of said body.

24. The system of claim 20 wherein said domesticated animal is selected from the group consisting of: mammals and avians.

25. The system of claim 20 wherein said domesticated animal comprises a pet.

26. The system of claim 25 wherein said pet is selected from the group consisting of: canines, felines and birds.

27. The system of claim 20 wherein said domesticated animal comprises a zoo animal.
